(19) United States
(12) Patent Application Publication
(10) Pub. No.: US 2015/0203540 A1
(43) Pub. Date: Jul. 23, 2015

(54) Cytoplasmic Tail Modifications to Boost Surface Expression and Immunogenicity of Envelope Glycoproteins

(71) Applicant: The Trustees of the University of Pennsylvania, Philadelphia, PA (US)

(72) Inventors: James A. Hoxie, Berwyn, PA (US); Jordan Andrea Polacchini-Oliviera, Blue Bell, PA (US); Angela Conde, Royersford, PA (US); Drew Weissman, Wynnewood, PA (US)

(21) Appl. No.: 14/417,228

(22) PCT Filed: Jul. 26, 2013
(86) PCT No.: PCT/US13/52248

§ 371 Date: Jan. 26, 2015

Related U.S. Application Data
(60) Provisional application No. 61/676,557, filed on Jul. 27, 2012.

(51) Int. Cl. C07K 14/005; A61K 39/21; C12N 7/00

(52) U.S. Cl. CPC C07K 14/005; C12N 7/00; A61K 39/21; C12N 2740/15034; C12N 2740/15022; C12N 2740/16134

(57) ABSTRACT

The invention provides compositions and methods for enhanced expression of a viral envelope protein. The invention provides a composition comprising a cytoplasmic tail modification to enhance surface expression of both HIV and non-HIV viral envelope proteins as wells as other membrane associated proteins resulting in increased immunogenicity.

### CROSS-REFERENCE TO RELATED APPLICATION

This application claims priority to U.S. Provisional Application Ser. No. 61/676,557, filed Jul. 27, 2012, the content of which is incorporated by reference herein in its entirety.

### STATEMENT REGARDING FEDERALLY SPONSORED RESEARCH OR DEVELOPMENT

This invention was made with government support under RO1 AI074362, R37 AI045378, RO1 AI084860, RO1 AI050484, RO1 AI090788, awarded by the National Institutes of Health (NIH). The government has certain rights in the invention.

### BACKGROUND OF THE INVENTION

Human immunodeficiency virus (HIV) entry is known to require an interaction of the viral envelope glycoprotein (Env) with CD4 and cellular chemokine receptors. HIV Env protein is produced as a precursor (gp160) that is subsequently cleaved into two parts, gp120 which binds CD4 and chemokine receptors, and gp41 which is anchored in the viral membrane and mediates membrane fusion. Differential use of chemokine receptors by HIV and SIV has largely explained differences in tropism among different isolates (Berger, 1997, AIDS 11:S3-S16; Hoffman and Doms, 1998, AIDS 12:S17-S26). While a number of chemokine receptors can be utilized by HIV or SIV (Deng et al., 1997, Nature 388:296-300; Choe et al., 1996, Cell 85, 1135-1148; Rucker et al., 1997, J. Virol. 71:8999-9007; Edinger et al., 1997, Proc. Natl. Acad. Sci. USA 94:14742-14747; Liao et al., 1997, J. Exp. Med. 185:2015-2023; Farzan et al., 1997, J. Exp. Med. 186:405411), CCRS and CXCR4 appear to be the principal coreceptors for HIV-1 (Zhang et. al., 1998, J. Virol. 72:9337-9344; Zhang et al., 1998, J. Virol. 72:9337-9344). Isolates of HIV that first establish infection target CD4+ T-lymphocytes using CCRS (Alkhatib et al., 1996, Science 272:1955-1958; Deng et al., 1996, Nature 381:661-666; Dragic et al., 1996, Nature 381:667-673; Doranz et al., 1996, Cell 85:1149-1158). In some patients these viruses can evolve to use the chemokine receptor CXCR4, which is associated with a more rapid progression to AIDS (Choe et al., 1996, Cell 85:1135-1148; Feng et al., 1996, Science 272:872-876; Connor et al., 1997, J. Exp. Med. 185:621-628).

HIV is particularly adept at evading humoral immune responses, a feature that likely contributes to the ability of this virus to establish a persistent infection. Although neutralizing antibodies are produced to viral envelope glycoproteins (Env), such antibodies are characteristically directed to hypervariable loops on gp120 (V1/V2 and V3), which can tolerate extensive genetic variation. These antibodies are in general “type specific” and easily circumvented by ongoing viral mutations.

The HIV-1 Env is the principal target of neutralizing antibodies and a key component in HIV vaccines that are designed to elicit protective humoral immune responses. Although immunogenic, attempts to generate neutralizing antibodies to Env have been limited by a number of barriers including: 1) a high degree of variability among different HIV isolates; 2) extensive glycosylation of external surfaces that renders these sites largely non-immunogenic (i.e., “the glycan shield”); 3) conformational flexibility that may prevent formation of neutralization epitopes to limit their recognition by humoral immune responses (i.e., “entropic masking”); 4) steric factors that restrict antibody access to conserved and functionally important domains; and 5) the immunodominance of non-neutralizing epitopes that likely serve as decoys to subvert responses from more relevant neutralization targets (Hoxie, 2010, Annu Rev Med 61:135-152; Mascola and Montefiori, 2010, Annu Rev Immunol 28:413-444). However, recent studies on HIV-1 infected individuals who have high titers of potent and broadly neutralizing antibodies have led to breakthroughs with these antibodies being cloned and characterized, and new insights have emerged as to how HIV can be neutralized by host humoral immune responses (Hoxie, 2010, Annu Rev Med 61:135-152; Mascola and Montefiori, 2010, Annu Rev Immunol 28:413-444; Scheid et al., 2009, Nature 458:636-640; Pancera et al., 2010, J Virol 84:8098-8110; Wu et al., 2010, Science 329:856-861; Zhou et al., 2010, Science 329:811-817; Walker et al., 2009, Science 326:285-289). There continue to be major gaps in the ability to translate this information on antigenicity to an understanding of what immunogens can elicit these antibodies. Unfortunately, while it has become clear that broadly neutralizing antibodies are highly desirable, to date no immunogen has been able to elicit them with any degree of efficiency (McMichael et al., 2003, Nat. Med. 9:874-80). It is therefore crucial for research to address why an infected host fails to produce these antibodies and how vaccines can be designed that will overcome this obstacle.

The ability of HIV-1 to escape the immune system has hindered development of efficacious vaccines to combat this important human pathogen. Thus, there is a long-felt and unfulfilled need for the development of effective vaccines and therapeutic modalities for HIV-1 infection in humans. The present invention meets these needs.

### SUMMARY OF THE INVENTION

The invention provides a hybrid molecule comprising a non-simian immunodeficiency virus (SIV) sequence segment encoding an envelope (Env) and a SIV sequence segment, wherein the SIV sequence segment comprises an SIV endocytosis motif or a variant, mutant, or fragment thereof, further wherein the hybrid molecule encodes an envelope protein comprising a membrane spanning domain (MSD).

In one embodiment, the non-SIV sequence segment comprises sequences of Envelope of a virus selected from the group consisting of HIV-1, influenza A, influenza B, Herpes Simplex Type 1, Herpes Simplex Type 2, Ebola, West Nile, Hepatitis C, Respiratory Syncytia Virus, Dengue, Chikungunya, rotavirus, EBV, CMV, Marburg, and any combination thereof.

In one embodiment, the non-SIV sequence segment comprises sequences of HIV-1 Env.

In one embodiment, the SIV endocytosis motif is GYRPV (SEQ ID NO: 1).

In one embodiment, the SIV endocytosis motif comprises a Y/I mutation thereby comprising GIRPV (SEQ ID NO: 3).

In one embodiment, the SIV endocytosis motif comprises a ΔGY mutation thereby comprising RPV (SEQ ID NO: 4).

In one embodiment, the SIV endocytosis motif comprises an R722G mutation thereby comprising GYGPV (SEQ ID NO: 5).

In one embodiment, the SIV sequence segment comprises the sequence of QGYRPVFSSPPSY (SEQ ID NO: 6).

In one embodiment, the SIV sequence segment comprises a S727P mutation thereby comprising the sequence of QGYRPVFSPPPSY (SEQ ID NO: 7).

In one embodiment, the hybrid molecule further comprises a stop codon that truncates the tail of the envelope protein.

In one embodiment, the stop codon that truncates the tail of the envelope protein is positioned after the SIV sequence segment.

In one embodiment, the stop codon that truncates the tail of the envelope protein is positioned before the start of the Tat/Rev 2^{nd} exon of the HIV-1 envelope protein.

In one embodiment, the sequence is a nucleotide sequence.

In one embodiment, the sequence is an amino acid sequence.

The invention provides a vector comprising the sequence of the hybrid molecule of the invention.

The invention provides a host cell comprising the sequence of hybrid molecule of the invention.

The invention provides an immunogenic composition comprising the sequence of the hybrid molecule of the invention.

The invention provides an antibody or antigen binding fragment thereof that specifically binds the hybrid molecule of the invention.

The invention provides a pharmaceutical composition comprising the hybrid molecule of the invention and a pharmaceutically acceptable carrier.

The invention provides a method of generating an immune reaction in a mammal comprising administering an immunogen-stimulating amount of the hybrid molecule of the invention to a mammal, wherein the hybrid molecule encodes an envelope protein comprising a membrane spanning domain (MSD).

The invention provides a method for preventing a subject from becoming infected with HIV-1, the method comprising administering to the subject in need thereof a prophylactically effective amount of a composition comprising the hybrid molecule of the invention, wherein the hybrid molecule encodes an envelope protein comprising a membrane spanning domain (MSD), so as to thereby prevent the subject from becoming infected with HIV-1.

The invention provides a method for treating a subject infected with HIV-1, the method comprising administering to the subject in need thereof an effective amount of a composition comprising the hybrid molecule of the invention, wherein the hybrid molecule encodes an envelope protein comprising a membrane spanning domain (MSD), so as to thereby treat the subject from becoming infected with HIV-1.

The invention provides a method for enhancing expression of an envelope protein in a cell, the method comprising expressing the hybrid molecule of the invention in a cell, so as to thereby enhance expression of the envelope protein in the cell.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an image depicting sequences of the Env cytoplasmic tail for SIVmac and HIV-1 having the membrane spanning domains (MSD), the GYxxØ endocytosis motif (GYRPV for SIVmac, SEQ ID NO: 1; GYSPL for HIV-1, SEQ ID NO: 2), and the approximate start site for the second exons of Tat and Rev (in alternate reading frames). The chimeric HIV-1/SIV Env containing the indicated segment from SIVmac, a stop codon plus the Tyr→Ile mutation produced a large increase in surface expression, similar to what was seen for the SIV Env. Thus, high surface expression of an HIV-1 Env can be engineered by introducing this SIV segment along with the indicated mutations (designated “Y/I” and “F-stop”).

FIG. 2, comprising FIGS. 2A through 2C, is a series of images depicting enhanced surface expression of SIV Envs having modifications in their cytoplasmic tails. FIG. 2A is an image depicting sequences from the proximal Env cytoplasmic tail of SIVmac239 as represented in FIG. 1. The indicated mutations were introduced: “delta GY” to ablate the GYxxØ endocytosis signal and a stop codon proximal to the start sites for the second exons of Tat and Rev in alternate reading frames. FIG. 2B is an image demonstrating that Envs were transfected into 293T cells and the levels of surface Env were quantified by FACS using the anti-SIV gp120 antibody 7D3. Surface levels of Env were low for the parental (239 wt) Env and were minimally affected by the two mutations individually. However, when a Tyr→Ile mutation (Y/I), which also ablated the GYRPV signal, was introduced in combination with the stop codon, a large increase (˜8 fold) was observed over SIV wt Env. FIG. 2C is an image depicting FACS histograms for the “239 stop Y/I” Env in comparison to 239 wt. An isotype control stain of 293T cells for the mAb used in this experiment is also shown.

FIG. 3, comprising FIGS. 3A and 3B, is a series of images showing enhanced surface expression of HW-1 R3A Env containing an SIV cytoplasmic tail segment. (FIG. 3A) Sequences are shown from the membrane spanning domain and proximal Env cytoplasmic tails of SIVmac and a set of mutants based on the HIV-1 R3A isolate. In the first set (HIV-1), mutations are introduced 1) to truncate the SIV tail (“F-stop”) to remove more distal endocytosis signals; and 2) to introduce a Y/I mutation that ablates the GYxxØ endocytosis signal (GYSPL; SEQ ID NO: 2). In the second set (HIV-1/SIV) the indicated segment in the SIVmac cytoplasmic tail is introduced ±F-stop or Y/I mutations. (FIG. 3B) Envs were transfected into 293T cells and the levels of surface Env quantified by FACS using the anti-gp120 antibody 2G12. As shown, surface levels of R3A-based Envs are low for the parental (wt) Env and markedly increased by introducing the SIV segment with the F-stop and Y/I mutations. Without wishing to be bound by any particular theory, it is believed that this effect results from: 1) ablation of the proximal GYxxØ endocytosis signal (GYRPV), which down-regulates surface Env; 2) removal of endocytosis signals that are distal to the F-stop mutation; and 3) introduction of a positive regulator of Env surface expression contained within the SIV segment.

FIG. 4, comprising FIGS. 4A and 4B, is a series of images depicting enhanced surface expression of HIV-1 JRFL Env containing an SIV cytoplasmic tail segment. FIG. 4A is an image depicting sequences from the proximal Env cytoplasmic tails of SIVmac and HIV-1 JRFL with regions represented as in FIG. 1. A set of chimeras were created by introducing the indicated region of SIVmac into the HIV-1 tail alone and with the “F-stop” and “Y/I” mutations individually or in combination. FIG. 4B is an image demonstrating that Envs were transfected into 293T cells and the levels of surface Env quantified by FACS as in FIG. 3. It was observed that surface levels of JRFL were low for the parental (wt) Env and were unaffected by introducing the SIV segment with or without the F-stop or Y/I mutations when added individually. However, it was observed that for the HIV-1 R3A Env (FIG. 3), when both of these mutations were introduced a large (>16 fold) increase was observed over JRFL-wt.

FIG. 5 demonstrates Env surface expression in human dendritic cells. Human dendritic cells were derived from peripheral blood monocytes by culturing 6-7 days in IL-4 and GM-CSF and transfected by electroporation with: 1) wildtype HIV-1 R3A Env; or 2) R3A containing the SIV segment shown in FIG. 5 (SIV); a tyrosine to isoleucine mutation in the GYRPV (SEQ ID NO: 1) motif (Y/I); and a premature stop codon (F-stop) shown in FIG. 5. For this experiment, Envs were subcloned into the vector pVax for optimal expression in dendritic cells. As shown, Env expression was markedly increased by the changes in the Env cytoplasmic tail.

FIG. 6, comprising FIGS. 6A and 6B, is a series of images showing additional enhancement of surface expression of HIV-1 R3A Env following the introduction of mutations from SIVmac239ΔGY-infected rhesus macaques. (FIG. 6A) Sequences are shown for parental HIV-1/R3A and SIVmac239 membrane spanning domain and proximal cytoplasmic tail with the GYxxØ motif and the “SIV segment” noted in FIG. 5. Below, sequences are shown for HIV-1 Env constructs containing the SIV segment plus the indicated point mutations that emerged in rhesus macaques infected with the “ΔGY” mutant (i.e. R722G and S727P). Also shown is the premature termination codon (F-stop) described in the text, and the ΔGY mutation within the GYxxØ motif. (FIG. 6B) Envs were transfected into 293T cells and the levels of surface Env quantified by FACS using the anti-gp120 antibody 2G12. The fold increases in Env surface expression relative to wiltype HIV-1/R3A Env are shown for Envs depicted in the Top Panel. While an increase was seen with the introduction of the SIV, Y/I and F-stop mutations, levels were markedly increased (8-10 fold) when changes from ΔGY-infected animals were also incorporated (*). Without wishing to be bound by any particular theory, it is believed that the additional increase results from the effects of changes positively selected for in vivo that increase Env expression on virions to compensate for an assembly defect caused by the ΔGY mutation. While not restoring a recognizable endocytosis signal, these changes have been shown to restore Env content on virions and appear to correct the assembly defect caused by the ΔGY mutation.

### DETAILED DESCRIPTION OF THE INVENTION

The present invention relates to the discovery that the cytoplasmic tail of an envelope glycoprotein can be modified to increase surface expression thereof. This modification is broadly applicable to any membrane-based envelope including those with mutations in ectodomain that are designed to elicit qualitative differences in the immune response. Accordingly, the cytoplasmic tail modification of the present invention is broadly transferrable and can elicit immunogenicity against a variety of envelope immunogens in an animal model.

In one embodiment, the invention provides a hybrid or chimeric molecule that is derived from SIV and HIV. Preferably, the hybrid molecule is an HIV-1 Env containing a SIV segment comprising various mutations introduced in order to enhance expression of the Env.

In one embodiment, the hybrid molecule comprises a conserved endocytosis signal (i.e. GYxxØ, where G=glycine, Y=tyrosine; x=any amino acid; and Ø=an amino acid with a bulky hydrophobic side chain) or a variant, mutant, or fragment thereof that increases the steady state level of envelope surface expression. In another embodiment, the hybrid molecule includes a positive signal for surface expression. In this manner, the invention incorporates genetic modifications that increase Env surface expression by: 1) ablation of the proximal GYxxØ endocytosis signal, which down-regulates surface Env; and 2) introduction of positive regulator of Env surface expression contained within the SIV segment.

In one embodiment, the present invention provides a novel chimeric SIV/HIV envelope modification termed “SIV-Y/I F-stop” that significantly enhances cell surface expression of HIV-1 envelope glycoproteins (e.g., >3-fold compared to wildtype). It is hypothesized that the SIV/HIV envelope modification of the present invention will be useful for boosting expression of any membrane-based envelope and increasing the immunogenicity of the vaccines derived therefrom.

In one embodiment, the present invention provides a novel chimeric SIV/HIV molecule comprising one or more of an SIV segment, a Tyr to Ile mutation in GYxxØ (referred herein as “Y/I”), truncation of the SIV tail (e.g., “F-stop”), deletion of GY in GYxxØ (referred herein as “ΔGY”), an arginine to glycine mutation at amino acid 722 of the SIVmac239 molecular clone of the envelope gene (referred herein as the R722G mutation), and a serine to proline mutation at SIVmac239 amino acid position 727. The modifications of the invention allow for a heighten level of surface expression of HIV-1 envelope glycoproteins (e.g., >10-fold compared to wildtype). It is expected that the SIV/HIV envelope modifications of the present invention will be useful for boosting expression of any membrane-based HIV envelope glycoprotein and increasing the immunogenicity of the vaccines derived therefrom.

The invention is based on the discovery that the SIV tail modification produces a quantitatively greater increase in envelope surface expression than levels reported in the art. The SIV tail modification is transferrable to a variety of HIV-1 envelope immunogens. In one embodiment, the SIV tail modification is applicable to HIV and non-HIV viral proteins when there is a desire to produce a quantitative increase in their surface expression to augment humoral immune responses. For example, the SIV tail modification can be used to replace the RSV F protein cytoplasmic tail to increase the surface expression of RSV F protein. The modifications of the invention is also applicable to other envelope proteins including but are not limited to Respiratory Syncytial Virus, Hepatitis C, Dengue, Ebola, West Nile, Chikunguna, Herpes Simplex Types 1 and 2, rotavirus, EBV, CMV, Marburg, influenza, and the like.

In one embodiment, the invention provides a sequence useful for improving expression of a desired membrane-based envelope. The sequence comprises the GYxxØ (where G=glycine, Y=tyrosine, x=any amino acid, and Ø=a bulky hydrophobic amino acid) endocytosis motif wherein Tyr is mutated to Ile.

In another embodiment, the sequence for enhancing surface expression of an envelope protein comprises GYxxØ (where G=glycine, Y=tyrosine, x=any amino acid, and Ø=a bulky hydrophobic amino acid) endocytosis motif wherein Tyr is mutated to Ile and is truncated. Preferably, the truncation is a result of a stop codon inserted proximal to the start sites for the second exons of Tat and Rev (referred herein as the “SIV-Y/I-Fstop modification”).

The SIV-Y/I-Fstop modification as well as one or more modifications of the invention including, but is not limited to, an SIV segment, a Y/I mutation, an F-stop, a ΔGY mutation, a R722G mutation, and a S727P mutation, is applicable to any situation where it is desirable to improve expression of a desired membrane-based Env. Accordingly, the present invention provides a method for enhancing expression of any membrane-based envelope protein.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The following standard abbreviations are used throughout the specification to indicate specific amino acids: A=ala=alanine; R=arg=arginine; N=asn=asparagine; D=asp=aspartic acid; C=cys=cysteine; Q=gln=glutamine; E=glu=glutamic acid; G=gly=glycine; H=his=histidine; I=ile=isoleucine; L=leu=leucine; K=lys=lysine; M=met=methionine; F=phe=phenylalanine; P=pro=proline; S=ser=serine; T=thr=threonine; W=trp=tryptophan; Y=tyr=tyrosine; V=val=valine.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

As used herein, to “alleviate” a virus infection means reducing the severity of the symptoms of the disease or disorder.

As used herein the terms “alteration,” “defect,” “variation,” or “mutation,” refers to a mutation in the cytoplasmic tail of the cell surface expressed immunogen that affects the function, activity, expression (transcription or translation) or conformation of the polypeptide that it encodes. Mutations encompassed by the present invention can be any mutation that results in the enhancement of cell surface expression of the polypeptide.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

By the term “exogenous nucleic acid” is meant that the nucleic acid has been introduced into a cell or an animal using technology which has been developed for the purpose of facilitating the introduction of a nucleic acid into a cell or an animal.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, lentiviruses, adenoviruses, and adeno-associated viruses) that incorporate the recombinant polynucleotide.

A first region of an oligonucleotide “flanks” a second region of the oligonucleotide if the two regions are adjacent one another or if the two regions are separated by no more than about 1000 nucleotide residues, and preferably no more than about 100 nucleotide residues.

As used herein, the term “fragment” as applied to a nucleic acid, may ordinarily be at least about 18 nucleotides in length, preferably, at least about 24 nucleotides, more typically, from about 24 to about 50 nucleotides, preferably, at least about 50 to about 100 nucleotides, even more preferably, at least about 100 nucleotides to about 200 nucleotides, yet even more preferably, at least about 200 to about 300, even more preferably, at least about 300 nucleotides to about 400 nucleotides, yet even more preferably, at least about 400 to about 500, and most preferably, the nucleic acid fragment will be greater than about 500 nucleotides in length.

As applied to a protein, a “fragment” of a stimulatory or costimulatory ligand protein or an antigen, is about 6 amino acids in length. More preferably, the fragment of a protein is about 8 amino acids, even more preferably, at least about 10, yet more preferably, at least about 15, even more preferably, at least about 20, yet more preferably, at least about 30, even more preferably, about 40, and more preferably, at least about 50, more preferably, at least about 60, yet more preferably, at least about 70, even more preferably, at least about 80, and more preferably, at least about 100 amino acids in length amino acids in length.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are completely or 100% homologous at that position. The percent homology between two sequences is a direct function of the number of matching or homologous positions, e.g., if half (e.g., five positions in a polymer ten subunits in length) of the positions in two compound sequences are homologous then the two sequences are 50% identical, if 90% of the positions, e.g., 9 of 10, are matched or homologous, the two sequences share 90% homology. By way of example, the DNA sequences 5′ATTGCC3′ and 5′TATGGC3′ share 50% homology.

In addition, when the terms “homology” or “identity” are used herein to refer to the nucleic acids and proteins, it should be construed to be applied to homology or identity at both the nucleic acid and the amino acid sequence levels.

“HIV” refers to the human immunodeficiency virus. HIV includes, without limitation, HIV-1. HIV may be either of the two known types of HIV, i.e., HIV-1 or HIV-2. The HIV-1 virus may represent any of the known major subtypes or clades (e.g., Classes A, B, C, D, E, F, G, J, and H) or outlying subtype (Group 0). Also encompassed are other HIV-1 subtypes or clades that may be isolated.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

By the term “immunogenic dose,” as the term is used herein, is meant an amount of a polypeptide of the invention, or portion thereof, whether administered to a mammal as protein or as nucleic acid encoding the protein, which generates a detectable humoral and/or cellular immune response to the protein compared to the immune response detected in an otherwise identical mammal to which the protein is not administered. In one aspect, the dose is administered as Env protein, a gp120 polypeptide, or a fragment thereof. In another aspect, the dose is administered as a nucleic acid encoding the polypeptide of the invention.

“Immunizing” means generating an immune response to an antigen in a subject. This can be accomplished, for example, by administering a primary dose of an antigen, e.g., a vaccine, to a subject, followed after a suitable period of time by one or more subsequent administrations of the antigen or vaccine, so as to generate in the subject an immune response against the antigen or vaccine. A suitable period of time between administrations of the antigen or vaccine may readily be determined by one skilled in the art, and is usually on the order of several weeks to months. Adjuvant may or may not be co-administered.

“Instructional material,” as that term is used herein, includes a publication, a recording, a diagram, or any other medium of expression which can be used to communicate the usefulness of the composition and/or compound of the invention in the kit for effecting alleviating or treating the various diseases or disorders recited herein. Optionally, or alternately, the instructional material may describe one or more methods of alleviating the diseases or disorders in a cell or a tissue or a mammal, including as disclosed elsewhere herein.

The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide residues of the polynucleotide. It is understood that a portion of a polynucleotide may include every nucleotide residue of the polynucleotide.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

Preferably, when the nucleic acid encoding the desired protein further comprises a promoter/regulatory sequence, the promoter/regulatory is positioned at the 5′ end of the desired protein coding sequence such that it drives expression of the desired protein in a cell. Together, the nucleic acid encoding the desired protein and its promoter/regulatory sequence comprises a “transgene.”

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A “recombinant polypeptide” is one which is produced upon expression of a recombinant polynucleotide.

A “recombinant cell” is a cell that comprises a transgene. Such a cell may be a eukaryotic cell or a prokaryotic cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “transgene” means an exogenous nucleic acid sequence which exogenous nucleic acid is encoded by a transgenic cell or mammal

A “therapeutic” treatment is a treatment administered to a patient who exhibits signs of pathology for the purpose of diminishing or eliminating those signs and/or decreasing or diminishing the frequency, duration and intensity of the signs.

To “treat” a disease or disorder as the term is used herein, means to reduce the severity and/or frequency that at least one sign or symptom of the disease or disorder is experienced by an animal.

By the term “vector” as used herein, is meant any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector which is suitable as a delivery vehicle for delivery of a nucleic acid that encodes a protein and/or antibody of the invention, to the patient, or the vector may be a non-viral vector which is suitable for the same purpose.

Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a lentiviral vector, a recombinant adenovirus, a recombinant retrovirus, a recombinant adeno-associated virus, a recombinant avian pox virus, and the like (Cranage et al., 1986, EMBO J. 5:3057-3063; International Patent Application No. WO 94/17810, published Aug. 18, 1994; International Patent Application No. WO 94/23744, published Oct. 27, 1994). Examples of non-viral vectors include, but are not limited to, liposomes, polyamine derivatives of DNA, and the like.

By the term “vaccine,” as the term is used herein, is meant a compound which when administered to a human or veterinary patient, induces a detectable immune response, humoral and/or cellular, to an antigen, or a component(s) thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides methods and compositions for enhancing expression of a desired membrane-based envelope protein on a cell surface. The present invention is based on the discovery that cytoplasmic tail modifications can increase surface expression and immunogenicity of HIV envelope glycoproteins. The present invention is also based on the discovery that: 1) the HIV (and SIV) Env cytoplasmic tails contain a highly conserved endocytosis signal (i.e. GYxxØ, where G=glycine, Y=tyrosine; x=any amino acid; and Ø=an amino acid with a bulky hydrophobic side chain) that reduces the steady state expression level of Env on the cell surface; and 2) the SIV (but not the HIV) Env cytoplsamic tail contains an additional region flanking the GYxxØ endocytosis motif that positively regulates Env surface expression.

In one embodiment, the invention allows for enhancing expression of a desired membrane-based envelope protein (e.g., HIV Env) by generating a hybrid molecule between SIV and HIV wherein the increased Env expression is a result from: 1) ablation of the proximal GYxxØ endocytosis signal (e.g., GYRPV; SEQ ID NO: 1), which down-regulates surface Env; 2) removal of endocytosis signals that are distal to the F-stop mutation; and 3) introduction of an a positive regulator of Env surface expression contained within the SIV segment.

In one embodiment, the present invention provides a novel chimeric SIV/HIV comprising one or more of the following components: an SIV segment, a Tyr to Ile mutation in GYxxØ (referred herein as “Y/I”), truncation of the SIV tail (e.g., “F-stop”), deletion of GY in GYxxØ (referred herein as “ΔGY”), an arginine to glycine mutation at amino acid 722 of SIVmac or otherwise a mutation of the arginine in the GYRPV (SEQ ID NO: 1) endocytosis motif to a glycine (referred herein as R722G mutation), and a serine to proline mutation at amino acid 727 SIVmac (referred herein as S727P mutation).

In one embodiment, the cytoplasmic tail modification is the SIV-Y/I-Fstop modification which comprises the combination of a Tyr→Ile mutation in the GYxxØ endocytosis signal and a stop codon that is inserted immediately proximal to the start sites for the second exons of Tat and Rev in the HIV gene (i.e. F-stop). In another embodiment, the cytoplasmic tail modification includes the combination of one or more of a SIV segment, ΔGY, R722G mutation, S727P mutation, and F-stop (a stop codon that is inserted immediately proximal to the start sites for the second exons of Tat and Rev in the HIV gene). Based on the disclosure presented herein, cytoplasmic tail modifications play a predominant role in the expression levels of any membrane-based envelope protein.

In one embodiment, the invention provides an improved method of increasing the expression of an envelope protein. This platform can be generally applicable to any envelop-based vaccine where an envelope immunogen is presented on a cell membrane. In one embodiment, this approach is applicable to augment surface expression of non-HIV viral envelopes as well as other membrane-associated proteins that are targeted by vaccines.

In one embodiment, the present invention relates to a polynucleotide composition that provides enhanced efficiency in the expression of a protein or polypeptide in a cell (i.e., resulting in an increase in the level of the protein or polypeptide encoded by the polynucleic acid). The invention also includes methods for preparing the composition of the invention. In particular, the invention includes an isolated polynucleotide sequence that is capable of enhanced gene expression over a corresponding wild-type polynucleotide sequence. The ability to enhance gene expression is applicable to any setting in which it is desirable to express a gene.

The present invention contemplates embodiments directed to any gene that is poorly expressed or any gene for which improved levels of protein expression is desirable for in vivo and/or in vitro uses.

### HIV-1 Envelope Proteins

The invention relates to compositions and methods to increase surface expression of envelope proteins. In one embodiment, the invention encompasses an isolated or modified HIV-1 envelope protein that expresses epitopes which bind broadly cross-reactive neutralizing antibodies. Several classes of broadly neutralizing antibodies have been described including those reactive with the CD4 binding site, the coreceptor binding site, conformational determinants often involving variable loops V 1/V2 and/or V3 and conserved glycosylation sites, and a membrane proximal epitope on gp41. With the exception of the broadly neutralizing antibodies to CD4-inducible epitopes (i.e. epitopes that require CD4 binding to be exposed and/or formed), all epitopes are expressed on native trimers present on the cell surface and/or on virions. The isolated HIV-1 envelope proteins of the present invention express native trimers on the cell surface and do not require CD4 or coreceptor binding to be recognized. The invention therefore includes an HIV-1 envelope protein or fragment thereof comprising epitopes that are expressed on native trimers and as such are capable of binding to broadly cross reactive neutralizing antibody. In one embodiment, the epitope encompasses a component of the three dimensional structure of an HIV-1 envelope protein that is displayed regardless of whether or not the HIV-1 envelope protein is bound to a cell surface receptor. In one embodiment, these epitopes are linear amino acid sequences from a modified HIV-1 envelope protein. These epitopes contain amino acid sequences that correspond to amino acid sequences in epitopes that in most HIV envelope proteins are only transiently expressed during binding to a cell surface receptor. Nonetheless, the three dimensional structures are displayed on the protein surface in the absence of the envelope protein binding to a cell surface receptor. HIV-1 envelope proteins containing these epitopes are associated with a broadly cross-reactive neutralizing antibody response in humans.

HIV-1 envelope proteins containing modifications in the primary amino acid sequence result in enhanced surface expression of the envelope and associated epitopes which induce a neutralizing antiserum. Such modifications confer increased expression of the envelop protein and thereby the ability to induce desirable neutralizing antibody response both in vivo and in vitro. Central to this view is the recognition that the modifications described herein also impart increased surface expression of HIV-1 envelope proteins on human dendritic cells, which are critical in presenting antigen in the context of generating a humoral immune response. Such alterations include, but are not limited to, modifying the HIV-1 envelope to comprise a segment from the SIV cytoplasmic tail in the analogous position in the HIV-1 tail, thereby incorporating the desired SIV element to increase envelope surface expression.

In one embodiment, the hybrid HIV/SIV molecule of the invention comprises one or more of the following modifications to the cytoplasmic tail: an SIV segment, Y/I, F-stop, ΔGY, R722G, and S727P. In another embodiment, when the hybrid molecule of the invention comprises the Y/I cytoplasmic tail modification, the hybrid molecule does not comprise the ΔGY cytoplasmic tail modification. Similarly, when the hybrid molecule comprises the ΔGY cytoplasmic tail modification, the hybrid molecule does not comprise the Y/I cytoplasmic tail modification. This is because the Y that is deleted in the ΔGY modification is substituted with an Ile in Y/I modification.

In one embodiment, the hybrid HIV/SIV cytoplasmic tail comprises a Tyr to Ile mutation in the GYxxØ endocytosis signal in the proximal cytoplasmic tail of the envelope (e.g., referred elsewhere herein as the “SIV-Y/I”). In some instances, it is desirable to truncate the SIV tail of the HIV/SIV hybrid envelop (e.g., referred elsewhere herein as the “F-stop”). Accordingly, the invention provides an envelope glycoprotein that can be modified to contain the SIV-Y/I-Fstop cytoplasmic tail which results in a higher surface expression of the envelope and thereby a higher and more durable anti-envelop antibody titer.

In another embodiment, the cytoplasmic tail modification includes the combination of one or more of a SIV segment, ΔGY, R722G mutation, S727P mutation, and F-stop (a stop codon that is inserted proximal to the start sites for the second exons of Tat and Rev in the HIV gene).

The envelope proteins of the invention include the full length envelope protein wherein one or more epitope sites have been modified, and fragments thereof containing one or more of the modified epitope sites. In one embodiment, one or more amino acid residues are deleted while in another embodiment, one or more of these sites are substituted with another amino acid which alters the conformation of the epitope.

### Nucleic Acid Molecules

The present invention further includes an isolated nucleic acid molecule that encodes the isolated or modified HIV-1 envelope protein, or fragments thereof, that contain one or more of the modified epitopes, preferably in isolated form. As used herein, “nucleic acid” is defined as RNA or DNA that encodes a protein or peptide as defined above, is complementary to a nucleic acid sequence encoding such peptides, hybridizes to nucleic acid molecules that encode the isolated or modified HIV-1 envelope proteins across the open reading frame under appropriate stringency conditions, or encodes a polypeptide that shares at least about 75% sequence identity, preferably at least about 80%, more preferably at least about 85%, and even more preferably at least about 90% or even 95% or more identity with the isolated or modified HIV-1 envelope proteins.

The isolated nucleic acid of the invention further includes a nucleic acid molecule that shares at least 80%, preferably at least about 85%, and more preferably at least about 90% or 95% or more identity with the nucleotide sequence of a nucleic acid molecule that encodes an isolated or modified HIV-1 envelope protein, particularly across the open reading frame. Specifically contemplated are genomic DNA, cDNA, mRNA and antisense molecules, as well as nucleic acids based on alternative backbones or including alternative bases whether derived from natural sources or synthesized. Such nucleic acids, are defined further as being novel and unobvious over any prior art nucleic acid molecule including one that encodes, hybridizes under appropriate stringency conditions, or is complementary to a nucleic acid molecule encoding a protein according to the present invention.

Homology or identity at the nucleotide or amino acid sequence level is determined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference), recommended for query sequences over 85 in length (nucleotide bases or amino acids).

For blastn, the scoring matrix is set by the ratios of M (i.e., the reward score for a pair of matching residues) to N (i.e., the penalty score for mismatching residues), wherein the default values for M and N are +5 and -4, respectively. Four blastn parameters were adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The present invention further includes fragments of the isolated nucleic acid molecule which fragments encode contain the desired modification (i.e., modification of one or more amino acids in the selected epitope) in the envelope protein. As used herein, a fragment of an encoding nucleic acid molecule refers to a small portion of the entire protein coding sequence. The size of the fragment will be determined by the intended use. For example, if the fragment is chosen so as to encode an active portion of the protein (i.e., a selected monoclonal antibody epitope or modification of such an epitope as described herein), the fragment will need to be large enough to encode the functional regions of the protein (i.e., epitopes). For instance, a fragment which encodes a peptide corresponding to a predicted antigenic region may be prepared. On the other hand, if the fragment is to be used as a nucleic acid probe or PCR primer, then the fragment length is chosen so as to obtain a relatively small number of false positives during probing/priming.

Fragments of the isolated nucleic acid molecule of the present invention (i.e., synthetic oligonucleotides) that are used to synthesize sequences encoding proteins of the invention, can easily be synthesized by chemical techniques, for example, the phosphotriester method of Matteucci et al. (1981) J. Am. Chem. Soc. 103, 3185-3191 or using automated synthesis methods. In addition, larger DNA segments can readily be prepared by well-known methods, such as synthesis of a group of oligonucleotides that define various modular segments of the gene, followed by ligation of oligonucleotides to build the complete modified gene.

The isolated nucleic acid molecule of the present invention may further be modified so as to contain a detectable label for diagnostic and probe purposes. A variety of such labels is known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention. Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

### Gene Modification

The present invention further provides DNA molecules that have been subjected to molecular manipulation in situ. Methods for generating DNA molecules are well known in the art, for example, see Sambrook et al. (2001) Molecular Cloning; A Laboratory Manual, Cold Spring Harbor Laboratory Press. In the preferred DNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the DNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 (BioRad), pPL and pKK223 (Pharmacia).

Expression vectors compatible with eukaryotic cells, preferably those compatible with vertebrate cells, can also be used to form DNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies Inc.), pTDT1 (ATCC), the vector pCDM8 described herein, and the like eukaryotic expression vectors.

Eukaryotic cell expression vectors used to construct the DNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. Alternatively, the selectable marker can be present on a separate plasmid, and the two vectors are introduced by co-transfection of the host cell, and selected by culturing in the appropriate drug for the selectable marker. The present invention further provides host cells transformed with a nucleic acid molecule that encodes a protein of the present invention. The host cell can be either prokaryotic or eukaryotic.

Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH-3T3) available from the ATCC as CRL 1658, baby hamster kidney cells (BHK), and the like eukaryotic tissue culture cell lines. Any prokaryotic host can be used to express a DNA molecule encoding a protein of the invention. The preferred prokaryotic host is E. coli.

Transformation of appropriate cell hosts with a DNA molecule of the present invention is accomplished by well-known methods that typically depend on the type of vector used and host system employed. With regard to transformation of prokaryotic host cells, electroporation and salt treatment methods are typically employed, see, for example, Cohen et al. (1972) Proc. Natl. Acad. Sci. USA 69, 2110; and Sambrook et al. (2001) Molecular Cloning—A Laboratory Manual, Cold Spring Harbor Laboratory Press. With regard to transformation of vertebrate cells with vectors containing DNA, electroporation, cationic lipid or salt treatment methods are typically employed, see, for example, Graham et al. (1973) Virol. 52, 456; Wigler et al. (1979) Proc. Natl. Acad. Sci. USA 76, 1373-1376.

Successfully transformed cells, i.e., cells that contain a DNA molecule of the present invention, can be identified by well-known techniques including the selection for a selectable marker. For example, cells resulting from the introduction of a DNA molecule of the present invention can be cloned to produce single colonies. Cells from those colonies can be harvested, lysed and their DNA content examined for the presence of the DNA using well-known methods in the art or the proteins produced from the cell can be assayed via an immunological method.

In accordance with the invention, numerous vector systems for expression of the isolated or modified HIV-1 envelope protein may be employed. For example, one class of vectors utilizes DNA elements which are derived from animal viruses, such as bovine papilloma virus, polyoma virus, adenovirus, vaccinia virus, baculovirus, retroviruses (RSV, MMTV or MoMLV), Semliki Forest virus or SV40 virus. Additionally, cells which have stably integrated the DNA into their chromosomes may be selected by introducing one or more markers which allow for the selection of transfected host cells. The marker may provide, for example, prototrophy to an auxotrophic host, biocide resistance, (e.g., antibiotics) or resistance to heavy metals such as copper or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals. The cDNA expression vectors incorporating such elements include those described by Okayama (1983) Mol. Cell. Biol. 3, 280-289.

The vectors used in the subject invention are designed to express high levels of HIV-1 envelope proteins in cultured eukaryotic cells as well as efficiently secrete these proteins into the culture medium. In one embodiment, the targeting of the HIV-1 envelope proteins into the culture medium is accomplished by fusing in-frame to the mature N-terminus of the HIV-1 envelope protein the tissue plasminogen activator (tPA) prepro-signal sequence.

The HIV-1 envelope protein may be produced by (a) transfecting a mammalian cell with an expression vector encoding the HIV-1 envelope protein; (b) culturing the resulting transfected mammalian cell under conditions such that HIV-1 envelope protein is produced; and (c) recovering the HIV-1 envelope protein from the cell culture media or the cells themselves.

Once the expression vector or DNA sequence containing the constructs has been prepared for expression, the expression vectors may be transfected or introduced into an appropriate mammalian cell host. Various techniques may be employed to achieve this, such as, for example, protoplast fusion, calcium phosphate precipitation, electroporation or other conventional techniques. In the case of protoplast fusion, the cells are grown in media and screened for the appropriate activity.

Methods and conditions for culturing the resulting transfected cells and for recovering the HIV-1 envelope protein so produced are well known to those skilled in the art, and may be varied or optimized depending upon the specific expression vector and mammalian host cell employed.

In accordance with the claimed invention, the preferred host cells for expressing the HIV-1 envelope protein of this invention are mammalian cell lines. Mammalian cell lines include, for example, monkey kidney CV1 line transformed by SV40 (COS-7); human embryonic kidney line 293 (HEK293); baby hamster kidney cells (BHK); Chinese hamster ovary-cells-DHFR (CHO); Chinese hamster ovary-cells DHFR(DXB11); monkey kidney cells (CV1); African green monkey kidney cells (VERO-76); human cervical carcinoma cells (HELA); canine kidney cells (MDCK); human lung cells (W138); human liver cells (HepG2); mouse mammary tumor (MMT 060562); mouse cell line (C127); and myeloma cell lines.

Other eukaryotic expression systems utilizing non-mammalian vector/cell line combinations can be used to produce the envelope proteins. These include, but are not limited to, baculovirus vector/insect cell expression systems and yeast shuttle vector/yeast cell expression systems.

Methods and conditions for purifying HIV-1 envelope proteins from the culture media are provided in the invention, but it should be recognized that these procedures can be varied or optimized as is well known to those skilled in the art.

The HIV-1 envelope proteins or fragments thereof of the present invention may also be prepared by any known synthetic techniques. Conveniently, the proteins may be prepared using standard solid-phase synthetic techniques.

### Vaccine

The present invention provides the preparation of an envelope protein, which can be administered in a vaccine. The envelope protein can express an epitope that elicits an immune response when administered to a mammal Preferably, the envelope protein has the same structure as the native structure found on the surface of the virus.

The present invention includes methods of generating antibodies in a subject comprising administering one or more of the proteins, polypeptides and nucleic acids of the present invention, in an amount sufficient to induce the production of the antibodies. In preferred embodiments, the methods produce a highly potent, rapid neutralizing antibody response. The methods may be used for treatment of or for prevention of infection by HIV-1.

When used in a vaccine, the isolated or modified HIV-1 envelope protein, or fragment thereof, may be in the form of a “subunit” vaccine. Such a vaccine offers significant advantages over traditional vaccines in terms of safety and cost of production. Subunit vaccines may be less immunogenic than whole-virus vaccines, and it is possible that adjuvants with significant immunostimulatory capabilities may also be required so that the vaccine reaches its full potential.

Currently, adjuvants approved for human use in the United States include aluminum salts (alum). These adjuvants have been useful for some vaccines including hepatitis B, diphtheria, polio, rabies, and influenza. Other useful adjuvants include Complete Freund's Adjuvant (CFA), Incomplete Freund's Adjuvant (IFA), Muramyl dipeptide (MDP), synthetic analogues of MDP, N-acetylmuramyl-L-alanyl-D-isoglutamyl-L-alanine-2-[1,2-dipalmitoyl-s-glycero-3-(hydroxyphosphoryloxy)]ethylamide (MTP-PE) and compositions containing a degradable oil and an emulsifying agent, wherein the oil and emulsifying agent are present in the form of an oil-in-water emulsion having oil droplets substantially all of which are less than one micron in diameter.

The formulation of a vaccine of the invention should contain an effective amount of the desired envelope protein or fragment thereof. That is, included in the invention is an amount of an envelope protein which, optionally in combination with an adjuvant, induces a specific immunological response when administered to mammal so as to provide a beneficial effect to the mammal Such beneficial effect may include protection from subsequent exposure to whole virus, a diminution of virus load in the subject, and the like. In addition, the mammal may produce specific antibodies which can be used for diagnostic or therapeutic purposes.

The vaccine of the invention is also useful for the prevention or treatment of HIV-1 infection. The invention is particularly directed to the prevention and therapeutic use of the vaccine of the invention in a human. Often, more than one administration may be required to bring about the desired prophylactic or therapeutic effect; the exact protocol (dosage and frequency) can be established by standard clinical procedures.

The vaccine is administered in any conventional manner which introduces the vaccine into the mammal, usually by injection. For oral administration, the vaccine is administered in a form similar to those used for the oral administration of other proteinaceous materials. As discussed elsewhere herein, the precise amounts and formulations for use in either prevention or therapy can vary depending on the circumstances of the inherent purity and activity of the envelope protein, any additional ingredients or carriers, the method of administration and the like.

By way of non-limiting illustration, the vaccine dosages administered typically can be, with respect to the envelope protein, a minimum of about 0.1 mg/dose, more typically a minimum of about 1 mg/dose, and often a minimum of about 10 mg/dose. The maximum dosages are typically not as critical. Usually, however, the dosage may be no more than 500 mg/dose, often no more than 250 mg/dose. These dosages can be suspended in any appropriate pharmaceutical vehicle or carrier in sufficient volume to carry the dosage. Generally, the final volume, including carriers, adjuvants, and the like, typically may be at least 0.1 ml, more typically at least about 0.2 ml. The upper limit is governed by the practicality of the amount to be administered, generally no more than about 0.5 ml to about 1.0 ml.

In an alternative format, vaccine may be prepared as in a vector format, which vector expresses the HIV-1 envelope protein, or fragment thereof, in the host mammal Any available vaccine vector may be used, including Venezuelan equine encephalitis virus (see U.S. Pat. No. 5,643,576), poliovirus (see U.S. Pat. No. 5,639,649), pox virus (see U.S. Pat. No. 5,770,211) and vaccina virus (see U.S. Pat. Nos. 4,603,112 and 5,762,938). Alternatively, naked nucleic acid encoding the protein or fragment thereof may be administered directly to effect expression of the antigen in the mammal (see U.S. Pat. No. 5,739,118).

Different HIV-1 envelope proteins or fragments thereof may be used as immunogens in various combinations with each other. For example, an envelope protein that is expected to induce antibodies against one or more epitopes in gp41, may be used in combination with an envelope glycoprotein that is expected to induce antibodies against epitopes in gp120. Additional envelope glycoproteins may be combined in the immunization regimen, particularly envelope proteins that induce antibodies against additional epitopes or that represent variant forms of the same epitopes expressed by different subtypes of HIV-1. Different segments of these envelope glycoproteins may be used, such as gp120 from one strain of HIV-1 and gp41 from other strains of HIV-1.

### Enhancing Expression of Any Membrane-Based Envelope

The present invention has broad application outside expression of HIV-1 envelope protein. This is because the present invention relates to the discovery that the cytoplasmic tail of an envelope glycoprotein can be modified to increase surface expression thereof. This modification is broadly applicable to any membrane-based envelope including those with mutations in ectodomain that are designed to elicit qualitative differences in the immune response. Accordingly, the cytoplasmic tail modification of the present invention is broadly transferrable and can elicit immunogenicity against a variety of envelope immunogens in an animal model.

The present invention provides isolated nucleic acid molecules (polynucleotide molecules) comprising nucleotide base sequences that enhance expression of a desired envelope protein. Such nucleic acid molecules comprise a cytoplasmic tail modification (e.g., SIV-Y/I-Fstop modification or one or more modifications of the invention including but is not limited to an SIV segment, a Y/I mutation, an F-stop, a ΔGY mutation, a R722G mutation, and a S727P mutation) described elsewhere herein and a polynucleotide encoding a desired envelope protein. The presence of the cytoplasmic tail modification on an expression vector enhances the level of expression of the envelope protein encoded by the polynucleotide that reside on the expression vector as compared to the level of expression in the cytoplasmic modification. The cytoplasmic tail modification of the invention on an expression vector may enhance expression of one or more envelop protein whether encoded on separate corresponding nucleic molecules or whether encoded on a single polycistronic nucleic acid molecule present on the expression vector. The cytoplasmic tail modification may be used to enhance the level of expression of an envelope protein using both stable expression systems and transient expression systems as described elsewhere herein.

In a preferred embodiment, the invention provides an expression vector comprising at least the cytoplasmic tail modification described elsewhere herein and a polynucleotide encoding a desired envelope protein. Such a cytoplasmic tail modification provides enhanced (elevated) levels of expression in an appropriate host cell of at least one envelope protein encoded on the expression vector compared to the level of expression in the host cell carrying the same expression vector lacking the cytoplasmic tail. Expression vectors useful in the invention include any nucleic acid vector molecule that can be engineered to encode and express one or more envelope proteins in an appropriate (homologous) host cell.

In another embodiment, the invention provides a host cell that contains an expression vector comprising the cytoplasmic tail modification and a gene that directs the expression of at least one envelope protein in the host cell. A host cell may be a eukaryotic or prokaryotic host cell. Preferred eukaryotic host cells for use in the invention include, without limitation, mammalian host cells, plant host cells, fungal host cells, eukaryotic algal host cells, protozoan host cells, insect host cells, and fish host cells. More preferably, a host cell useful in the invention is a mammalian host cell, including, but not limited to, a Chinese hamster ovary (CHO) cell, a COS cell, a Vero cell, an SP2/0 cell, an NS/0 myeloma cell, a human embryonic kidney (HEK 293) cell, a baby hamster kidney (BHK) cell, a HeLa cell, a human B cell, a CV-1/EBNA cell, an L cell, a 3T3 cell, a HEPG2 cell, a PerC6 cell, and an MDCK cell. Particularly preferred is a CHO cell that can be treated with a standard methotrexate treatment protocol to amplify the copy number of genes on an expression vector inserted into the host cell. Fungal cells that may serve as host cells in the invention include, without limitation, Ascomycete cells, such as Aspergillus, Neurospora, and yeast cells, particularly yeast of a genus selected from the group consisting of Saccharomyces, Pichia, Hansenula, Schizosaccharomyces, Kluyveromyces, Yarrowia, and Candida. Preferred yeast species that may serve as host cells for expression of recombinant proteins according to the invention include, but are not limited to, Saccharomyces cerevisiae, Hansenula polymorpha, Kluyveromyces lactis, Pichia pastoris, Schizosaccharomyces pombe, and Yarrowia lipolytica. Prokaryotic host cells that may be used for expressing recombinant proteins according to the invention include, without limitation, Escherichia coli, serovars of Salmonella enterica, Shigella species, Wollinella succinogenes, Proteus vulgaris, Proteus mirabilis, Edwardsiella tarda, Citrobacter freundii, Pasteurella species, Haemophilus species, Pseudomonas species, Bacillus species, Staphyloccocus species, and Streptococcus species. Other cells that may be used as host cells for expression of recombinant proteins according to the invention include protozoan cells, such as the trypanosomatid host Leishmania tarentolae, and cells of the nematode Caenorhaditis elegans.

Polynucleotides as described herein, vectors comprising a cytoplasmic tail modification described herein, and host cells comprising such vectors comprising a cytoplasmic tail modification as described herein may be used in a variety methods related to expression of envelope proteins of interest.

In one embodiment, the invention provides a method of enhancing expression of a protein of interest in a host cell comprising the step of inserting into a host cell a an expression vector that comprises the cytoplasmic tail modification and a sequence that encodes and directs the synthesis of the envelope protein of interest in the host cell and culturing the host cell under conditions promoting expression of the envelope protein.

An envelope protein whose expression may be enhanced by incorporation of the cytoplasmic tail modification of the invention may be any envelope protein (including peptides, polypeptides, and oligomeric proteins) for which a functional envelope gene(s) can be engineered into a nucleic acid vector molecule for expression in an appropriate host cell.

### Expression Cassette

In other related aspects, the invention includes an expression cassette that is useful for improving expression of a desired envelope gene in a cell. In one embodiment, the cassette comprises one or more of the following elements that are operably linked from 5′ to 3′: 1) sequence corresponding to the desired membrane spanning domain (MSD), 2) sequence corresponding to the GYxxØ endocytosis motif or a mutation, variant, or fragment thereof, and 3) a stop codon that truncates the tail of envelope protein.

In another embodiment, the cassette comprises one or more of the following elements that are operably linked from 5′ to 3′: 1) sequence corresponding to the desired membrane spanning domain (MSD), 2) sequence corresponding to the GYxxØ endocytosis motif or a mutation thereof, 3) ΔGY, 4) R722G mutation, 5) S727P mutation and 6) F-stop (a stop codon that is inserted proximal to the start sites for the second exons of Tat and Rev in the HIV gene).

In one embodiment, the sequence corresponding to the GYxxØ endocytosis motif comprises the GYxxØ endocytosis motif from SIV, wherein the sequence is GYRPV (SEQ ID NO: 1). In another embodiment, the GYxxØ endocytosis motif from SIV comprises a mutation where Tyr is mutated to Ile.

In one embodiment, the stop coding that truncates the tail of the envelope protein flanks the start of the Tat/Rev 2^{nd} exon of the envelope protein.

In any event, the expression cassette for improved expression of a desired envelope gene may be operably linked to a nucleic acid comprising a promoter/regulatory sequence such that the nucleic acid is preferably capable of directing expression of the envelope protein encoded by the nucleic acid corresponding to the expression cassette.

A promoter sequence is said to be “operably linked” to a coding DNA sequence if the two are situated such that the promoter DNA sequence influences the transcription of the coding DNA sequence. For example, if the coding DNA sequence codes for the production of a protein, the promoter DNA sequence would be operably linked to the coding DNA sequence if the promoter DNA sequence affects the expression of the protein product from the coding DNA sequence. For example, in a DNA sequence comprising a promoter DNA sequence physically attached to a coding DNA sequence in the same chimeric construct, the two sequences are likely to be operably linked.

The DNA sequence associated with the regulatory or promoter DNA sequence may be heterologous or homologous, that is, the inserted sequences may be from a different species than the recipient cell. In either case, the DNA sequences, vectors and cells of the present invention are useful for directing transcription of the associated DNA sequence so that the mRNA transcribed or the protein encoded by the associated DNA sequence is efficiently expressed.

Promoters are positioned 5′ (upstream) to the sequences that they control. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art and demonstrated herein with multiple copies of regulatory elements, some variation in this distance can occur.

The coding sequence may be derived in whole or in part from a bacterial genome or episome, eukaryotic genomic, mitochondrial or plastid DNA, cDNA, viral DNA, or chemically synthesized DNA. It is possible that a coding sequence may contain one or more modifications in coding region which may affect the biological activity or the chemical structure of the expression product, the rate of expression, or the manner of expression control. Such modifications include, but are not limited to, mutations, insertions, deletions, rearrangements and substitutions of one or more nucleotides. The coding sequence may constitute an uninterrupted coding sequence or it may include one or more introns, bounded by the appropriate functional splice junctions. The coding sequence may be a composite of segments derived from a plurality of sources, naturally occurring or synthetic. The structural gene may also encode a fusion envelope protein, so long as the experimental manipulations maintain functionality in the joining of the coding sequences.

In preparing the constructs of this invention, the various DNA fragments may be manipulated, so as to provide for the DNA sequences in the proper orientation and, as appropriate, in the proper reading frame. Adapters or linkers may be employed for joining the DNA fragments or other manipulations may be involved to provide for convenient restriction sites, removal of superfluous DNA, removal of restriction sites, or the like.

For expression of the desired envelope gene, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

### Host Cells for Enhanced Production of Polypeptides of Interest

The present invention provides a polynucleotide that contains a coding sequence for an envelope protein that is operably linked to a promoter sequence and possibly other transcriptional regulatory sequences to direct proper transcription of the coding sequence into messenger RNA (mRNA) and that also comprises any of a variety of translation regulatory sequences that may be necessary or desired to direct proper translation of the mRNA into the desired protein in the intended host cell. A translational start codon (e.g., ATG) and a ribosome binding site are typically required in the mRNA for translation to occur in prokaryotic and eukaryotic cells. Other translation regulatory sequences that may also be employed, depending on the host cell, include, but are not limited to, an RNA splice site and a polyadenylation site.

The cytoplasmic tail modification of the invention serves to enhance the level of expression of an envelope protein encoded by one or more functional genes that reside on the expression vector as compared to the level of expression in the absence of the cytoplasmic tail modification in a host cell.

A host cell can be any cell, i.e., any eukaryotic or prokaryotic cell, into which a vector molecule can be inserted. According to the present invention, preferred host cells are eukaryotic or prokaryotic cells, including, but not limited to, animal cells (e.g., mammalian, bird, and fish host cells), plant cells (including eukaryotic algal cells), fungal cells, bacterial cells, and protozoan cells. Host cells useful in the invention may be of any genetic construct, but are preferably haploid or diploid cells. Preferred mammalian host cells useful in the invention include, without limitation, a Chinese hamster ovary (CHO) cell, a COS cell, a Vero cell, an SP2/0 cell, an NS/0 myeloma cell, a human embryonic kidney (HEK 293) cell, a baby hamster kidney (BHK) cell, a HeLa cell, a human B cell, a CV-1/EBNA cell, an L cell, a 3T3 cell, an HEPG2 cell, a PerC6 cell, and an MDCK cell. A preferred insect cell is Sf9. Fungal cells that may serve as host cells in the invention include, without limitation, Ascomycete cells, such as Aspergillus, Neurospora, and yeast cells, particularly yeast of the genera Saccharomyces, Pichia, Hansenula, Schizosaccharomyces, Kluyveromyces, Yarrowia, and Candida. Particularly preferred yeast fungal species that may serve as host cells for expression of recombinant proteins are Saccharomyces cerevisiae, Hansenula polymorpha, Kluyveromyces lactis, Pichia pastoris, Schizosaccharomyces pombe, and Yarrowia lipolytica. Preferred prokaryotic cells that may serve as host cells in the invention include, without limitation, Escherichia coli, serovars of Salmonella enterica, Shigella species, Wollinella succinogenes, Proteus vulgaris, Proteus mirabilis, Edwardsiella tarda, Citrobacter freundii, Pasteurella species, Haemophilus species, Pseudomonas species, Bacillus species, Staphyloccocus species, and Streptococcus species. Other cells that may be useful host cells for the expression of recombinant proteins according to the invention include protozoans, such as the trypanosomatid host Leishmania tarentolae, and cells of the nematode Caenorhaditis elegans. Various expression vectors are available for use in the aforementioned cells.

There are a variety of means and protocols for inserting vector molecules into cells including, but not limited to, transformation, transfection, cell or protoplast fusion, use of a chemical treatment (e.g., polyethylene glycol treatment of protoplasts, calcium treatment, transfecting agents such as LIPOFECTIN™ and LIPOFECTAMINE™ transfection reagents available from Invitrogen (Carlsbad, Calif.), use of various types of liposomes, use of a mechanical device (e.g., nucleic acid coated microbeads), use of electrical charge (e.g., electroporation), and combinations thereof. It is within the skill of a practitioner in the art to determine the particular protocol and/or means to use to insert a particular vector molecule described herein into a desired host cell.

Methods for transferring nucleic acid sequence information from one vector or other nucleic acid molecule to another are not limiting in the present invention and include any of a variety of genetic engineering or recombinant nucleic acid techniques known in the art. Particularly preferred transfer techniques include, but are not limited to, restriction digestion and ligation techniques, polymerase chain reaction (PCR) protocols (utilizing specific or random sequence primers), homologous recombination techniques (utilizing polynucleotide regions of homology), and non-homologous recombination (e.g., random insertion) techniques. Nucleic acid molecules containing a specific sequence may also be synthesized, e.g., using an automated nucleic acid synthesizer, and the resulting nucleic acid product then incorporated into another nucleic acid molecule by any of the aforementioned methodologies.

Employing genetic engineering technology necessarily requires growing recombinant host cells (e.g., transfectants, transformants) under a variety of specified conditions as determined by the requirements of the cells and the particular cellular state desired by the practitioner. For example, a host cell may possess (as determined by its genetic disposition) certain nutritional requirements, or a particular resistance or sensitivity to physical (e.g., temperature) and/or chemical (e.g., antibiotic) conditions. In addition, specific culture conditions may be necessary to regulate the expression of a desired gene (e.g., the use of inducible promoters), or to initiate a particular cell state (e.g., yeast cell mating or sporulation). These varied conditions and the requirements to satisfy such conditions are understood and appreciated by practitioners in the art.

The vectors harboring the gene of interest and the cytoplasmic tail modification described herein can be introduced into an appropriate host cell by any means known in the art. For example, the vector can be transfected into the host cell by calcium phosphate co-precipitation, by conventional mechanical procedures such as microinjection or electroporation, by insertion of a plasmid encased in liposomes, and by virus vectors. These techniques are all well-known and routinely practiced in the art, e.g., Brent et al., Current Protocols in Molecular Biology, John Wiley & Sons, Inc. (ringbou ed., 2003); and Weissbach & Weissbach, Methods for Plant Molecular Biology, Academic Press, NY, Section VIII, pp. 42 1-463, 1988. Host cells which harbor the transfected vector can be identified and isolated using the selection marker present on the vector. Large numbers of recipient cells may then be grown in a medium which selects for vector-containing cells. These cells may be used directly or the expressed protein may be purified in accordance with conventional methods such as extraction, precipitation, chromatography, affinity methods, electrophoresis and the like. The exact procedure used will depend upon the specific protein produced and the specific vector/host expression system utilized.

In an embodiment, host cells for expressing the vectors are eukaryotic cells. Eukaryotic vector/host systems, and mammalian expression systems, allow for proper post-translational modifications of expressed mammalian proteins to occur, e.g., proper processing of the primary transcript, glycosylation, phosphorylation and advantageously secretion of expressed product. Therefore, eukaryotic cells such as mammalian cells can be the host cells for the protein of a polypeptide of interest. Examples of such host cell lines include CHO, BHK, HEK293, VERO, HeLa, COS, MDCK, NS0 and W138.

In some embodiments, engineered mammalian cell systems that utilize viruses or viral elements to direct expression of the protein of interest are employed. For example, when using adenovirus expression vectors, the coding sequence of a protein of interest along with the 28-codon tag may be ligated to an adenovirus transcription/translation control complex, e.g., the late promoter and tripartite leader sequence. This chimeric sequence may then be inserted into the adenovirus genome by in vitro or in vivo recombination. Insertion in a non-essential region of the viral genome (e.g., region E1 or E3) will result in a recombinant virus that is viable and capable of expressing the polypeptide of interest in infected hosts (e.g., see Logan & Shenk, Proc. Natl. Acad. Sci. USA 81:3655-3659, 1984). Alternatively, the vaccinia virus 7.5K promoter may be used. (e.g., see, Mackett et al., Proc. Natl. Acad. Sci. USA, 79:7415-7419, 1982; Mackett et al., J. Virol. 49:857-864, 1984; Panicali et al., Proc. Natl. Acad. Sci. USA, 79:4927-4931, 1982). Of particular interest are vectors based on bovine papilloma virus which have the ability to replicate as extrachromasomal elements (Sarver et al., Mol. Cell. Biol. 1:486, 1981). These vectors can be used for stable expression by including a selectable marker in the plasmid, such as the neo gene. Alternatively, the retroviral genome can be modified for use as a vector capable of introducing and directing the expression of the gene of interest in host cells (Cone & Mulligan, Proc. Natl. Acad. Sci. USA 8 1:6349-6353, 1984). High level expression may also be achieved using inducible promoters, including, but not limited to, the metallothionine IIA promoter and heat shock promoters.

The host cell for expression of the vectors can also be yeast. In yeast, a number of vectors containing constitutive or inducible promoters may be used. See, e.g., Brent et al., Current Protocols in Molecular Biology, John Wiley & Sons, Inc. (ringbou ed., 2003); and The Molecular Biology of the Yeast Saccharomyces, Strathem et al. (eds.), Cold Spring Harbor Press (1982). A constitutive yeast promoter such as ADH or LEU2 or an inducible promoter such as GAL may be used. Alternatively, vectors may be used which promote integration of foreign DNA sequences into the yeast chromosome.

In cases where plant expression vectors are used, the expression of a gene of interest may be driven by any of a number of promoters. For example, viral promoters such as the .sup.35S RNA and 19S RNA promoters of CaMV (Brisson et al., Nature 310.about.511-514, 1984) or the coat protein promoter to TMV (Takamatsu et al., EMBO J., 6:307-3 11, 1987) may be used. Alternatively, plant promoters such as the small subunit of RUBISCO (Coruzzi et al., EMBO J. 3:1671 1680, 1984; and Broglie et al., Science 224:838-843, 1984) or heat shock promoters (Gurley et al., Mol. Cell. Biol., 6:559-565, 1986) may be used.

Once the vector has been introduced into the appropriate host cells, the expressed protein may be purified in accordance with conventional methods such as extraction, precipitation, chromatography, affinity chromatography, electrophoresis and the like. The exact procedure used will depend upon both the specific protein produced and the specific expression system utilized. For long-term, high-yield production of proteins, stable expression is preferred. Rather than using expression vectors which contain origins of replication, host cells can be transformed with a vector that allows stable integration of the vector into the host chromosomes. Host cells with stably integrated polynucleotides that encode the protein of interest can grow to form foci which in turn can be cloned and expanded into cell lines. For example, following the introduction of foreign DNA, engineered cells may be allowed to grow for 1-2 days in an enriched media, and then switched to a selective media.

### Therapeutic Applications

An appropriate level of a protein in mammalian cells is a critical factor for inducing an immunological and/or therapeutic response, e.g., the use of the gene and its protein product as an immunogen, DNA vaccine, co-immunogen, adjuvant, carrier protein or vector, therapeutic agent, diagnostic agent, therapeutic, immuno-prophylactic, immuno-therapeutic, etc., The efficiency of a gene in expressing its protein product is a controlling factor in the attainment of appropriate levels of the protein in cells. Certain genes fail to provide appropriate protein levels in mammalian cells. The present invention is directed to improving the expression efficiency of such genes.

A vector for therapeutic expression of proteins can be constructed with the cytoplasmic tail modification (e.g., SIV-Y/I-Fstop modification or one or more modifications of the invention including but is not limited to an SIV segment, a Y/I mutation, an F-stop, a ΔGY mutation, a R722G mutation, and a S727P mutation) described elsewhere herein and a polynucleotide encoding a desired envelope protein. Other examples include vectors to be used in vaccines so that increased envelope protein production can be achieved.

In some embodiments, the translational enhancer elements and polynucleotides disclosed herein are used in the preparation of DNA vaccines. In order to produce increased envelope protein levels, the DNA vaccines can be generally comprised of an expression vector wherein expression of a vaccine envelope protein is enhanced by the presence of the cytoplasmic tail modification (e.g., SIV-Y/I-Fstop modification or one or more modifications of the invention including but is not limited to an SIV segment, a Y/I mutation, an F-stop, a ΔGY mutation, a R722G mutation, and a S727P mutation) of the invention. In some embodiments, the DNA vaccines can deliver and express a desired envelope protein in combination with other antigens. Other than sequences encoding the vaccine envelope protein, the DNA vaccine vector typically also includes a promoter for transcription initiation that is active in eukaryotic cells. Such DNA vaccine vectors can be generated in accordance with the methods well known in the art. For example, methods for making and using DNA vaccine for a given antigen are described in, e.g., Gurunathan et al., Ann. Rev. Immunol., 18:927, 2000; Krieg, Biochim. Biophys. Acta., 1489:107, 1999; Cichutek, Dev. Biol. Stand., 100:119, 1999; Davis, Microbes Infect., 1:7, 1999; and Leitner, Vaccine, 18:765, 1999.

A diverse array of vaccine envelope proteins can be expressed by the DNA vaccines. These include, e.g., HIV-1, influenza A and B, Herpes Simplex Type 1 and 2, Ebola, Hepatitis C, Respiratory Syncytia Virus, Dengue, and Chikungunya.

The DNA vaccines can be used to immunize any subject in need of prevention or protection against infection of a pathogen (e.g., HIV infection). Such subjects include humans and non-human animals such as rodents (e.g. mice, rats and guinea pigs), swine, chickens, ferrets, non-human primates. Methods of administering a DNA vaccine to a suitable subject are described in the art. See, e.g., Webster et al, Vacc., 12:1495-1498, 1994; Bernstein et al., Vaccine, 17:1964, 1999; Huang et al., Viral Immunol., 12:1, 1999; Tsukamoto et al., Virol. 257:352, 1999; Sakaguchi et al., Vaccine, 14:747, 1996; Kodihalli et al., J. Virol., 71: 3391, 1997; Donnelly et al., Vaccine, 15:865, 1997; Fuller et al., Vaccine, 15:924, 1997; Fuller et al., Immunol. Cell Biol., 75: 389, 1997; Le et al., Vaccine, 18:1893, 2000; Boyer et al., J. Infect. Dis., 181:476, 2000.

In addition to enhancing expression of the desired envelope protein by using the cytoplasmic tail modification (e.g., SIV-Y/I-Fstop modification or one or more modifications of the invention including but is not limited to an SIV segment, a Y/I mutation, an F-stop, a ΔGY mutation, a R722G mutation, and a S727P mutation) of the present invention, the DNA vaccine can also be formulated with an adjuvant. Suitable adjuvants that can be employed include, e.g., aluminum phosphate or aluminum hydroxyphosphate, monophosphoryl-lipid A, QS-21 saponin, dexamethasone, CpG DNA sequences, Cholera toxin, cytokines or chemokines. Such adjuvants enhance immunogenicity of the DNA vaccines. Methods of preparing such modified DNA vaccines are known in the art. See, e.g., Ulmer et al., Vaccine 18:18, 2000; Schneerson et al. J. Immunol. 147:2136-2140, 1991; Sasaki et al. Inf. Immunol. 65: 3520-3528, 1997; Lodmell et al. Vaccine 18:1059-1066, 2000; Sasali et al., J. Virol. 72:4931, 1998; Malone et al., J. Biol. Chem. 269:29903, 1994; Davis et al., J. Immunol. 15:870, 1998; Xin et al., Clin. Immunol., 92:90, 1999; Agren et al., Immunol. Cell Biol. 76:280, 1998; and Hayashi et al. Vaccine, 18: 3097-3105, 2000.

In some embodiments, provided are methods for enhancing expression of a therapeutic protein in the treatment of various diseases. In these methods, an expression vector harboring a cytoplasmic tail modification (e.g., SIV-Y/I-Fstop modification or one or more modifications of the invention including but is not limited to an SIV segment, a Y/I mutation, an F-stop, a ΔGY mutation, a R722G mutation, and a S727P mutation) of the present invention and expressing a desired envelope protein are transfected into target cells, ex vivo or in vivo, through the interaction of the vector and the target cell. The compositions are administered to a subject in an amount sufficient to elicit a therapeutic response in the subject. Such gene therapy procedures have been used to correct acquired and inherited genetic defects, cancer, and viral infection in a number of contexts. See, e.g., Anderson, Science 256:808-813, 1992; Nabel & Felgner, TIBTECH 11:211-217, 1993; Mitani & Caskey, TIBTECH 11:162-166, 1993; Mulligan, Science 926-932, 1993; Dillon, TIBTECH 11: 167-175, 1993; Miller, Nature 357:455-460, 1992; Van Brunt, Biotechnology 6:1149-1154, 1998; Vigne et al., Restorative Neurol. and Neurosci. 8:35-36, 1995; Kremer & Perricaudet, Br. Med. Bull. 51:31-44, 1995; Haddada et al., in Current Topics in Microbiology and Immunology (Doerfler & Bohm eds., 1995); and Yu et al., Gene Therapy 1: 13-26, 1994.

Various diseases and disorders are suitable for treatment with the therapeutic methods described herein. These include malignancies of the various organ systems, e.g., lung, breast, lymphoid, gastrointestinal, and genito-urinary tract. Also suitable for treatment are adenocarcinomas which include malignancies such as most colon cancers, renal-cell carcinoma, prostate cancer, non-small cell carcinoma of the lung, cancer of the small intestine, and cancer of the esophagus. An expression vector containing a cytoplasmic tail modification (e.g., SIV-Y/I-Fstop modification or one or more modifications of the invention including but is not limited to an SIV segment, a Y/I mutation, an F-stop, a ΔGY mutation, a R722G mutation, and a S727P mutation) of the invention is also useful in treating non-malignant cell-proliferative diseases such as psoriasis, pemphigus vulgaris, Behcet's syndrome, and lipid histiocytosis. Essentially, any disorder that can be treated or ameliorated with a therapeutic envelope protein is considered susceptible to treatment with an expression vector that expresses the therapeutic envelope protein at increased level due to the presence of the cytoplasmic tail modification (e.g., SIV-Y/I-Fstop modification or one or more modifications of the invention including but is not limited to an SIV segment, a Y/I mutation, an F-stop, a ΔGY mutation, a R722G mutation, and a S727P mutation) in the vector.

A large number of delivery methods can be used to practice the therapeutic methods described herein. These methods are all well known to those of skill in the art. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those described in, e.g., WO 91/17424 and WO 91/16024. Delivery can be to cells (ex vivo administration) or target tissues (in vivo administration).

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al. (Proc. Natl. Acad. Sci. USA. 92:9747-9751, 1995) reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

The expression vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the vector. Ex vivo cell transfection for diagnostics, research, or for gene therapy (e.g., via re-infusion of the transfected cells into the host organism) is well known to those of skill in the art. In an embodiment, cells can be isolated from the subject organism, transfected with a nucleic acid (gene or cDNA), and re-infused back into the subject organism (e.g., subject). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from subjects).

### Pharmaceutical Composition

The invention encompasses the preparation and use of pharmaceutical compositions comprising a composition useful for treatment of a disease, disorder, or condition associated with an envelope glycoprotein (e.g., HIV) disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically-acceptable carrier” means a chemical composition with which an appropriate inhibitor thereof, may be combined and which, following the combination, can be used to administer the appropriate inhibitor thereof, to a subject.

The pharmaceutical compositions useful for practicing the invention may be administered to deliver a dose of between about 0.1 ng/kg/day and 100 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate inhibitor thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLE

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Cytoplasmic Tail Modifications

Aside from barriers to antibody binding, there are also widely appreciated but less well-described quantitative difficulties in expressing HIV-1 envelope glycoproteins on a cell surface. When cells are infected by HIV or transduced with Env-containing expression vectors (e.g. DNA, RNA and viral vectors), the level of surface Env is typically low. For HIV-1 and the related simian immunodeficiency virus (SIV) Envs, it has been discovered that a potent GYxxØ endocytosis signal in the proximal cytoplasmic tail (where G=glycine, Y=tyrosine, x=any amino acid, and Ø=a bulky hydrophobic amino acid) reduces steady state levels of Env on the cell surface. This motif is highly conserved in all HW and SIV Envs. Without wishing to be bound by any particular theory, it is believed that the presence of the potent GYxxØ endocytosis signal in the proximal cytoplasmic tail suggests a mechanism that ensures this protein is either incorporated into budding viral particles or rapidly cleared from the cell surface via clathrin-dependent endocytosis (Ohno et al., 1995, Science 269:1872-1875; Berlioz-Torrent et al., 1999, J Virol 73:1350-1361; Boge et al., 1998, J Biol Chem. 273:15773-15778; Sauter et al., 1996, Journal of Cell Biology 132:795-811; Rowell et al., 1995, Journal of Immunology 155:473-488).

For both HW and SIV Envs there are additional endocytosis signals that are less well defined on more distal regions of the cytoplasmic tail (Sauter et al., 1996, Journal of Cell Biology 132:795-811; Rowell et al., 1995, Journal of Immunology 155:473-488; Bowers et al., 2000, Traffic 1:661-674; Byland, et al., 2007, Molec Biol

Cell 18(2):414-25). It is believed that the clearance mechanism could help the virus evade host humoral immune responses by reducing the susceptibility of virus-producing cells to direct antibody-mediated killing and to antibody-dependent cellular cytoxicity (ADCC) (Marsh et al., 1997, Trends in Biochemical Sciences 7:1-4). It is also possible that this mechanism could reduce the immunogenicity of Env-based vaccines, given that antibody responses in preclinical vaccine trials are typically weak and only transient. Interestingly, for the SIV Env it has been shown that truncation of the cytoplasmic tail, which occurs when SIVs are grown in human cells leaving only 16 amino acids (Kodama et al., 1989, Journal of Virology 63:4709-4714), combined with a mutation that ablates the GYxxØ endocytosis signal results in the massive over-expression of Env on the cell surface to levels >10-20 times that of the wildtype Env (Sauter et al., 1996, Journal of Cell Biology 132:795-811; LaBranche et al., 1994, Journal of Virology 68:5509-5522; LaBranche et al., 1995, Journal of Virology 69:5217-5227). Moreover, it has been shown in a murine model that cells expressing an SIV Env that contained these mutations and exhibited this high surface expression phenotype elicited antibodies that potently neutralized SIV and exhibited neutralization breadth among some heterologous SIV isolates (Edinger et al., 2000, J Virol 74:7922-7935). While these findings suggested that similar modifications in the HIV-1 cytoplasmic tail could be useful for an Env-based HIV vaccine, analogous mutations (i.e. a Tyr mutation to eliminate the GYxxØ signal or a premature truncation at a comparable position) produced a less impressive effect (<3 fold) increase in Env surface expression, highlighting apparent differences in trafficking signals between HIV-1 and SIV Envs.

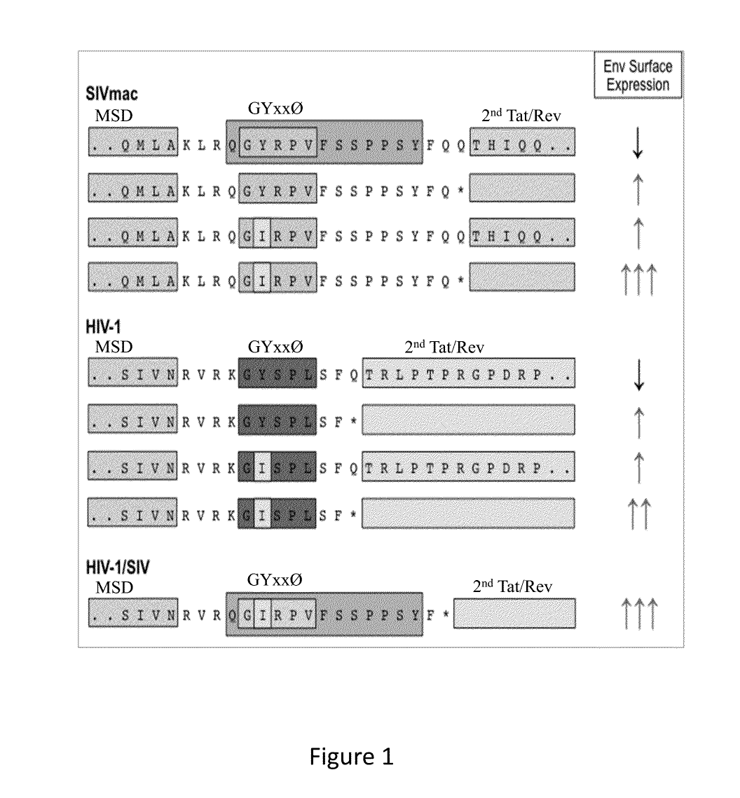

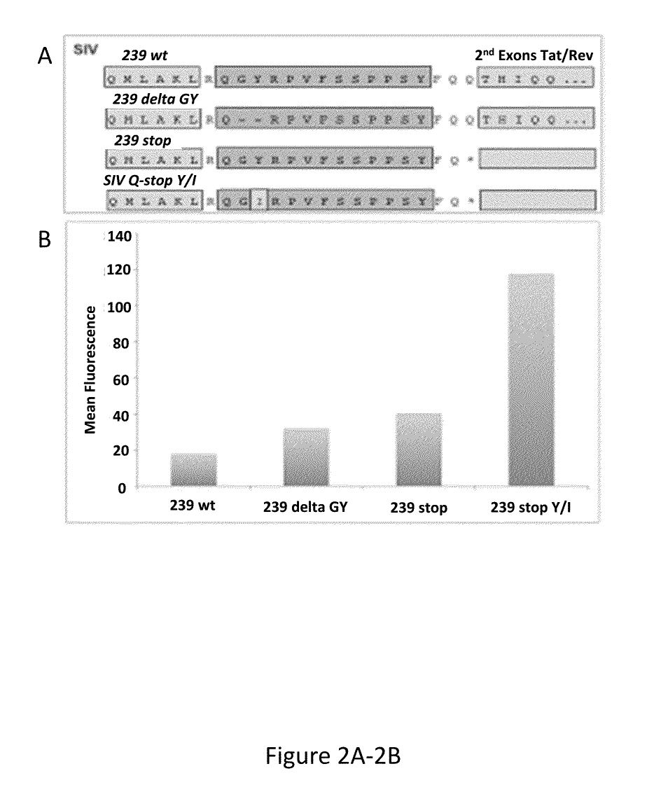

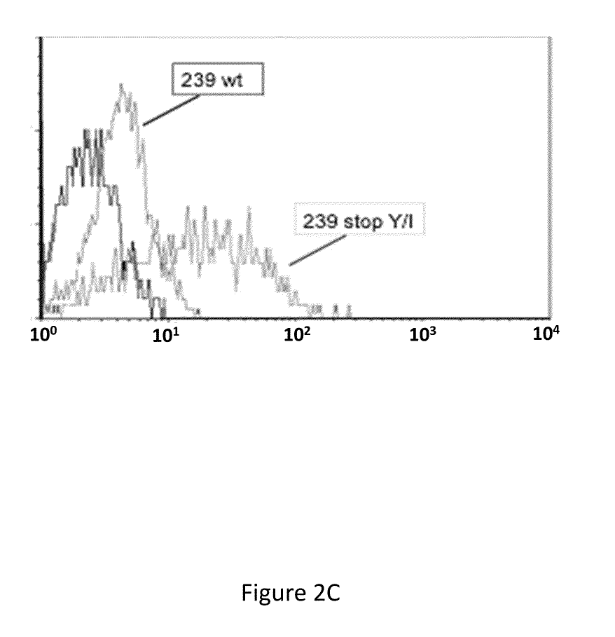

The results presented herein demonstrate that the SIV Env cytoplasmic tail has, in addition to the GYxxØ endocytosis motif that it shares with HIV-1, a region within the first 16 amino acids not present in HIV-1 that positively regulates Env expression on the cell surface (FIGS. 1 and 2). Therefore, for the SIV Env, its surface expression is regulated by a balance of determinants that can either reduce (i.e. via endocytosis signals) or enhance Env surface expression. In this context, differences in Env surface expression between SIV and HIV occur because HIV-1 lacks the SIV positive regulatory element.

Experiments were conducted to modify the HIV-1 Env by introducing a thirteen (13) amino acid segment from the SIV cytoplasmic tail in the analogous position in the HIV-1 tail, thereby incorporating the desired SIV element to increase Env surface expression. When this HIV/SIV Env chimera was further mutated to ablate the GYxxØ endocytosis motif (i.e. by a Tyr to Ile mutation) and truncated after the SIV segment, the level of Env expression was increased >10 fold over wildtype HIV-1 Env, similar to what was achieved for an SIV Env (FIG. 1).

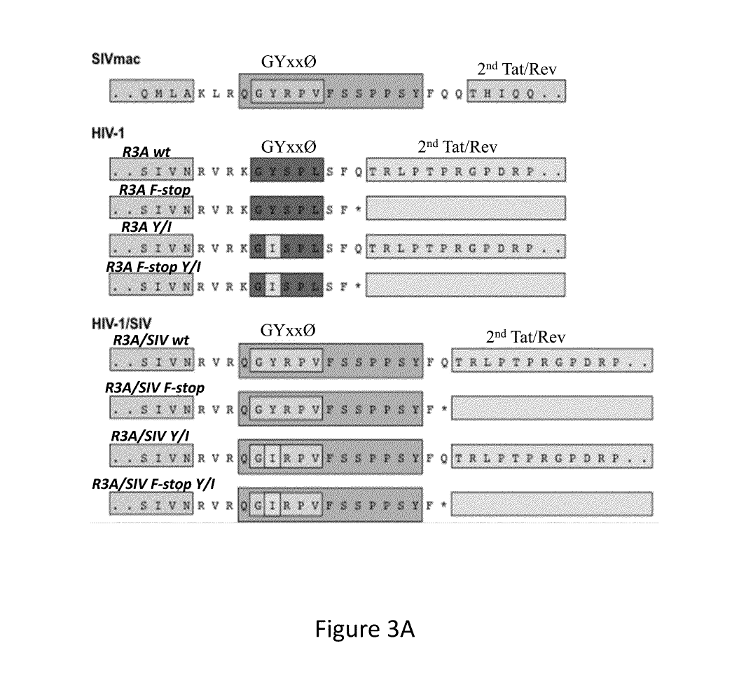

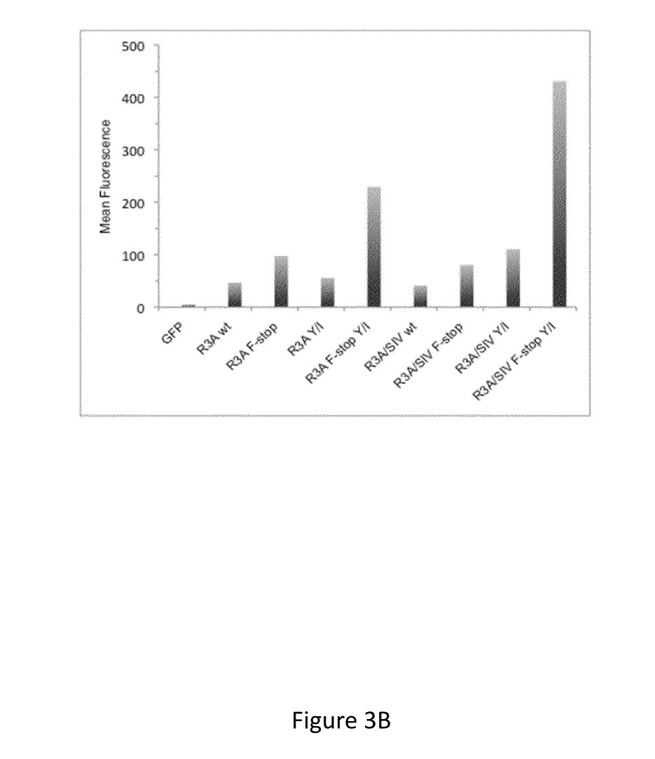

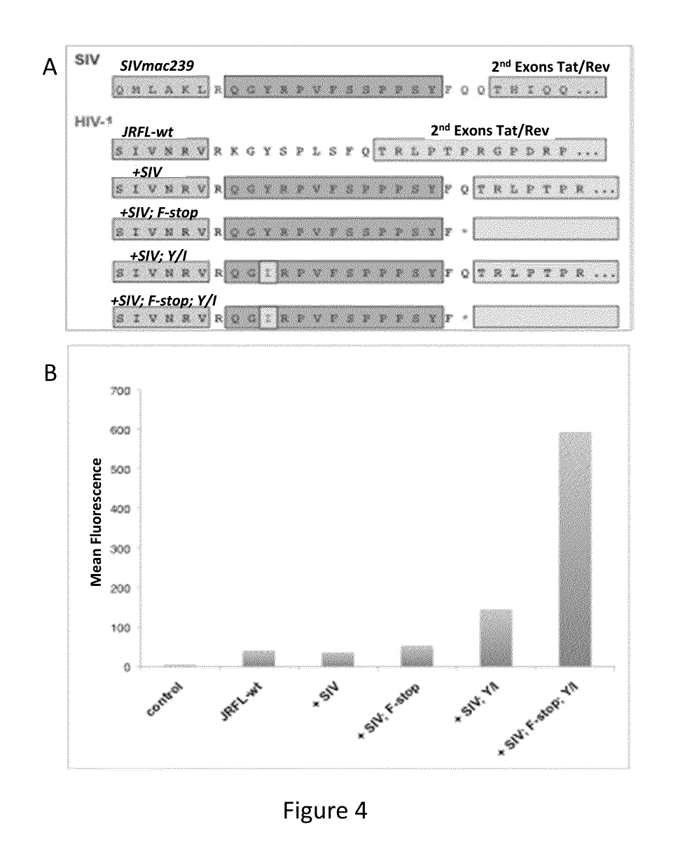

Briefly, various constructs were engineered to determine what factors attributed to the increased expression of Env. FIG. 1 shows the sequences of the Env cytoplasmic tail for SIVmac and HW-1 having the membrane spanning domains (MSD), the GYxxØ endocytosis motif (GYRPV (SEQ ID NO: 1) for SIVmac; GYSPL (SEQ ID NO: 2) for HIV-1), and the approximate start site for the second exons of Tat and Rev (in alternate reading frames). The HIV-1/R3A sequence is shown, but this region is conserved in most HIV-1 isolates. The relative levels of Env surface expression (by FACS) on transfected 293T cells is indicative with the characteristically low values indicated for SIVmac and HIV-1. For the SIVmac group, a stop codon flanking the start of the Tat/Rev 2^{nd} exon or the ablation of the GYxxØ signal by a Tyr→Ile mutation produced a slight (2-3 fold) increase in Env surface expression, whereas both mutations in combination produce a large (>10-20 fold) increase (FIG. 2). For HIV-1, only a slight increase in Env surface expression occured when similar mutations, either alone or in combination, are introduced (FIG. 3). However, for the chimeric HIV-1/SIV Env containing the indicated segment from SIVmac, a stop codon plus the Tyr→Ile mutation produced a large increase in surface expression, similar to what was seen for the SIV Env (FIGS. 3 and 4). Thus, high surface expression of an HIV-1 Env can be engineered by introducing this SIV segment along with the indicated mutations (designated “Y/I” and “F-stop”).

Without wishing to be bound by any particular theory, it is believed that HIV-1 Envs containing this mutated SIV segment that exhibit a high expression phenotype on the cell surface, will exhibit enhanced immunogenicity due to increased interactions with B cells. The results presented herein shown that this approach can be achieved in heterologous HIV-1 Envs (FIGS. 3 and 4). Briefly, FIG. 3 demonstrates enhanced surface expression of HW-1 R3A Env containing an SIV cytoplasmic tail segment. In a first set (HW-1), mutations were introduced to truncate the SIV tail at a position comparable to that shown in FIG. 2 (“F-stop”), to introduce a Y/I mutation that ablates the GYxxØ endocytosis signal (GYSPL, SEQ ID NO: 2), or both mutations. In the second set (HIV-1/SIV), the indicated segment in the SIV cytoplasmic tail was introduced±F-stop, Y/I or both mutations. The Envs were transfected into 293T cells and the levels of surface Env was quantified by FACS using the anti-gp120 antibody 2G12. It was observed that surface levels of R3A-based Envs were low for the parental (wt) Env and increased approximately 10 fold by introducing the SIV segment±the individual F-stop or Y/I mutations. Although modest effects were seen for some mutations, the “SIV-Fstop-Y/I” modification produced the greatest and most consistent increase. It is believed that this effect results from 1) ablation of the proximal GYxxØ endocytosis signal (GYRPV; SEQ ID NO: 1), which down-regulates surface Env; 2) removal of endocytosis signals that are distal to the F-stop mutation; and 3) introduction of a positive regulator of Env expression contained within the SIV segment (purple box).

In the next set of experiments, a set of chimeras were created by introducing the indicated region of SIVmac into the HW-1 tail alone and with the “F-stop” and “Y/I” mutations individually or in combination (FIG. 4). The various Envs were transfected into 293T cells and the levels of surface Env were quantified using FACS as in FIG. 3. It was observed that surface levels of Env cytoplasmic tails of HIV-1 JRFL were low for the parental (wt) Env and were unaffected by introducing the SIV segment with or without the F-stop or Y/I mutations when added individually. However, it was observed that for the HIV-1 R3A Env (FIG. 3), when both of these mutations were introduced a large (>16 fold) increase was observed over JRFL-wt.

The results presented herein show for two Clade B and one Clade A HIV-1 Envs that surface expression can be dramatically increased when the cytoplasmic tail is replaced by the mutagenized SIV segment discussed elsewhere herein, termed “SIV-Y/I-Fstop” (FIG. 2). These assays have been conducted on transfected 293T cells with Env surface expression being accessed by FACS. Without wishing to be bound by any particular theory, it is believed that this high surface expression can translate into an augmented humoral immune response in an animal model by comparing modified and unmodified Envs in a DNA prime/adenovirus boost protocol. The ability to increase Env surface expression is useful in the development of an Env-based vaccine that can be evaluated in humans.

The results presented herein addresses quantitative issues of Env expression on cells and demonstrate that when Env glycoproteins are modified to contain the SIV-Y/I-Fstop cytoplasmic tail, the result may be higher and more durable anti-Env antibody titers. As such, this modification is broadly applicable to any membrane-based Env including those with mutations in ectodomain that are designed to elicit qualitative differences in the immune response. For example, it is believed that immunogenicity of the respiratory syncytial virus (RSV) F protein, which is a validated target for protective immunity to RSV, can also be augmented by the SIV-Y/I-Fstop modification in its cytoplasmic tail. The cytoplasmic tail modification of the present invention is broadly transferrable and therefore can elicit immunogenicity in an animal model.

The results presented herein demonstrate that expression of HIV Envs can be enhanced following cytoplasmic tail modification. This platform can be generally applicable to any Env-based vaccine where an Env immunogen is presented on a cell membrane. Moreover, this approach may have even broader applicability to augment surface expression of non-HIV viral Envs or other membrane-associated proteins that are being targeted by vaccines.

### Example 2

### Mutations Emerging From In Vivo Non-Human Primate Studies Using ΔGY

The results presented herein provide a novel approach to augment the expression of HIV envelope glycoproteins (Env) on the cell surface, which is useful in increasing the immunogenicity of this protein in vaccines in the context for example a DNA or vectored immunogen. The HIV Env is the target to which neutralizing antibodies are directed and a key component of many vaccine candidates. The present invention is based on the discovery that: 1) the HIV (and SIV) Env cytoplasmic tails contain a highly conserved endocytosis signal (i.e. GYxxØ, where G=glycine, Y=tyrosine; x=any amino acid; and Ø=an amino acid with a bulky hydrophobic side chain) that reduces the steady state expression level of Env on the cell surface; and 2) the SIV (but not the HIV) Env cytoplsamic tail contains an additional region flanking the GYxxØ endocytosis motif that positively regulates Env surface expression.

It has been shown that when the region from SIV is engrafted on the HIV tail and the GYxxØ signal is ablated (with a Tyr to Ile mutation), the steady state level of Env surface expression can be increased, owing to the loss of the negative endocytosis signal and the inclusion of SIV's positive signal for surface Env expression. The maximal effect also requires a truncation of the cytoplasmic tail to remove more distal endocytosis signals, which have been shown to also down-modulate Env expression (see FIG. 3). As described elsewhere herein, the engineered increase in Env surface expression effect could be shown for several HIV Envs and therefor this approach can be used as a general strategy to increase Env presentation to antigen presenting cells and, as a result, immunogenicity. Given that Env is recognized as a protein that is poorly immunogenic in many vaccines, the present invention has the potential to address a fundamental problem in the HW vaccine field (i.e. poor expression and immunogenicity of Env-based vaccines).

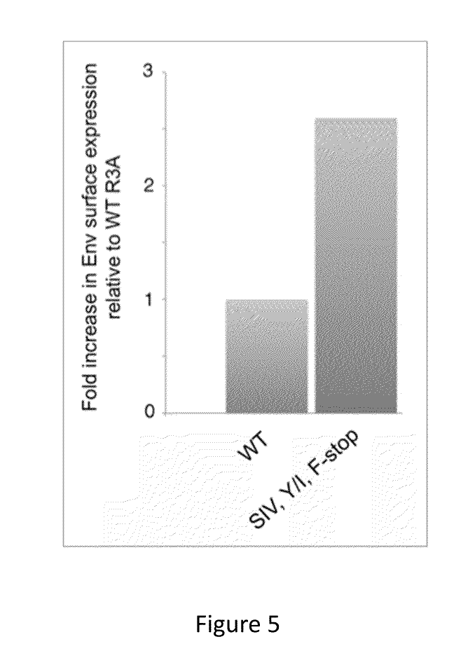

It has been demonstrated that surface expression of an HW-1 Env with the aforementioned tail modifications is also increased when expressed in human dendritic cells, which are the critical antigen presenting cells for humoral immune responses (see FIG. 5).

### Second Generation Mutations

To better understand the role of the highly conserved GYxxØ signal in pathogenesis, experiments were performed to evaluate the in vivo consequences of various mutations in this region (Fultz, et al., 2001, J Virol 75:278-291).

The present invention is based on ablating this signal by a GY deletion (i.e. within residues 719-724: QGYRPV→Q—RPV), creating the virus termed “ΔGY.” It was observed that ΔGY replicates to wildtype levels acutely, but exhibits a striking alteration in the anatomic distribution of virus with reduced to absent infection of gut lymphoid CD4+ T-cells, which are rapidly infected and profoundly depleted within the first month of infection. This loss of CD4 cells in this compartment is believed to contribute to a disruption of the epithelial barrier and the translocation of microbial products from the gut lumen to the systemic circulation. This microbial translocation has been proposed to drive the chronic immune activation that is typical of pathogenic SIV and HIV infection and viewed as a requirement for disease progression (Estes, et al., 2010, PLoS Pathog 6:e1001052; Douek, Det al., 2009, Annu Rev Med 60:471-484; Brenchley and Douek, 2008, Curr Opin HIV AIDS 3:356-361). Although ΔGY-infected animals developed a lower set point of plasma viral RNA as evidence of enhanced host control, they nonetheless progressed to disease, even without gut damage or microbial translocation.

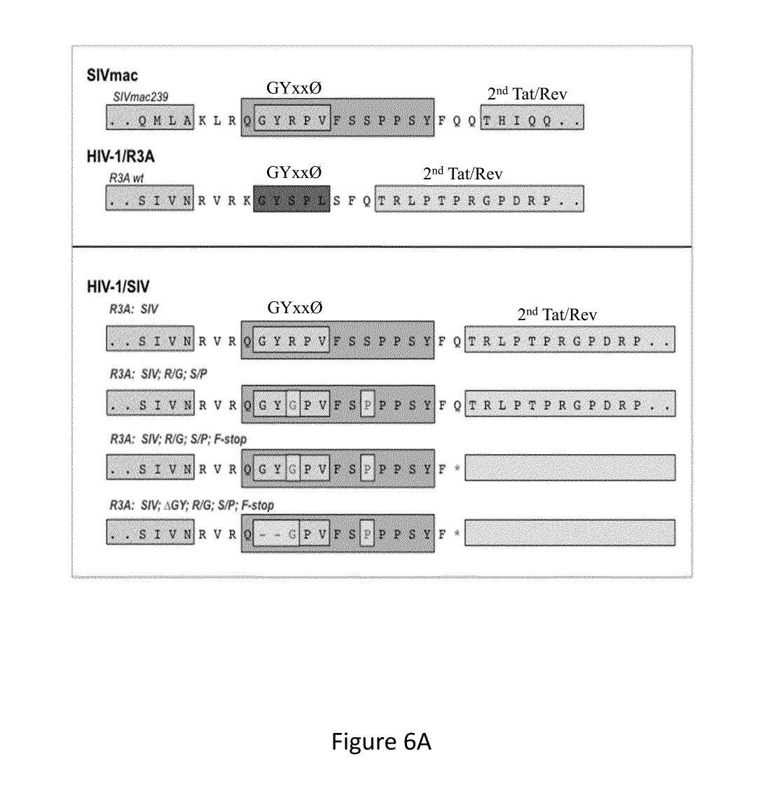

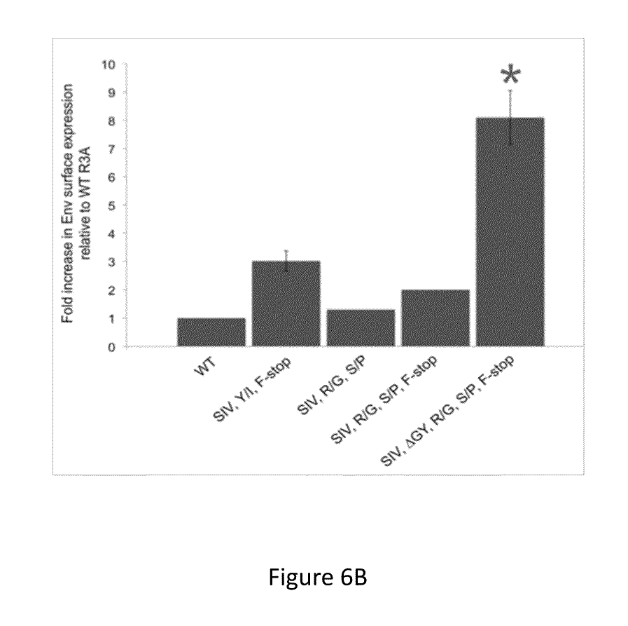

Interestingly, the ΔGY mutation has been shown to reduce the incorporation of Env on viral particles by 40-50% rendering viral particles more neutralization sensitive. Of relevance, animals that progressed to disease developed mutations flanking the ΔGY mutation that have been shown to compensate for ΔGY mutation by restoring Env content on virions even though they do not reconstitute a recognizable GYxxØ motif nor a recognizable endocytosis signal. These mutations include an Arg to Gly mutation at amino acid 722 (R722G) and a serine to proline mutation at position 727 (S727P). Remarkably, it has been shown that the R722G and S727P mutations when introduced into an HIV Env that contains the SIV segment and the ΔGY mutation increases surface Env expression to levels 3 fold higher than the first generation modifications (i.e. with the Y721I and the premature stop codon), and 8-10 fold greater than wildtype Env (FIG. 6).

In summary, the discovery that a segment from the SIV Env cytoplasmic tail when inserted into the HIV-1 tail can enable surface Env expression to be upregulated by selected mutations (initially Y721I and a premature stop codon) has now been enhanced to include the R722G and S727P mutations that emerged in vivo from the non-human primate studies with ΔGY. As a result of these findings, the mutations discussed herein can be incorporated in the Env cytoplasmic tail to augment the expression of HIV-1 Env vaccine candidates.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A hybrid molecule comprising a simian immunodeficiency virus (SIV) sequence segment and a non-SIV sequence segment encoding an envelope (Env), wherein the SIV sequence segment comprises an SIV endocytosis motif or a variant, mutant, or fragment thereof, and wherein the hybrid molecule encodes an envelope protein comprising a membrane spanning domain (MSD).

2. The hybrid molecule of claim 1, wherein the non-SIV sequence segment comprises at least one sequence of an Envelope of a virus selected from the group consisting of HW-1, influenza A, influenza B, Herpes Simplex Type 1, Herpes Simplex Type 2, Ebola, West Nile, Hepatitis C, Respiratory Syncytia Virus, Dengue, Chikungunya, rotavirus, EBV, CMV, Marburg, and any combination thereof.

3. The hybrid molecule of claim 1, wherein the non-SIV sequence segment comprises a sequence of HIV-1 Env.

4. The hybrid molecule of claim 1, wherein the SIV endocytosis motif is GYRPV (SEQ ID NO: 1).

5. The hybrid molecule of claim 1, wherein the SIV endocytosis motif is the mutant SIV endocytosis motif GIRPV (SEQ ID NO: 3).

6. The hybrid molecule of claim 1, wherein the SIV endocytosis motif is the ΔGY mutant SIV endocytosis motif RPV (SEQ ID NO: 4).

7. The hybrid molecule of claim 1, wherein the SIV endocytosis motif is the R722G mutant SIV endocytosis motif GYGPV (SEQ ID NO: 5).

8. The hybrid molecule of claim 1, wherein the SIV sequence segment comprises the sequence of QGYRPVFSSPPSY (SEQ ID NO: 6).

9. The hybrid molecule of claim 1, wherein the SIV sequence segment is the S727P mutant SIV sequence segment QGYRPVFSPPPSY (SEQ ID NO: 7).

10. The hybrid molecule of claim 1, further comprising a stop codon that truncates the tail of the envelope protein.

11. The hybrid molecule of claim 1, wherein the stop codon that truncates the tail of the envelope protein is positioned after the SIV sequence segment.

12. The hybrid molecule of claim 10, wherein the stop codon that truncates the tail of the envelope protein is positioned before the start of the Tat/Rev 2^{nd} exon of the HIV-1 envelope protein.

13. The hybrid molecule of claim 1, wherein the sequence is a nucleotide sequence.

14. The hybrid molecule of claim 1, wherein the sequence is an amino acid sequence.

15. A vector comprising the sequence of the hybrid molecule of claim 1.

16. A host cell comprising the sequence of hybrid molecule of claim 1.

17. An immunogenic composition comprising the sequence of the hybrid molecule of claim 1.

18. An antibody or antigen binding fragment thereof that specifically binds the hybrid molecule of claim 1.

19. A pharmaceutical composition comprising the hybrid molecule of claim 1 and a pharmaceutically acceptable carrier.

20. A method of generating an immune response in a mammal comprising administering an immunogen-stimulating amount of the hybrid molecule of claim 1 to a mammal, wherein the hybrid molecule encodes an envelope protein comprising a membrane spanning domain (MSD).

21. A method for preventing a subject from becoming infected with HIV-1, the method comprising administering to the subject in need thereof a prophylactically effective amount of a composition comprising the hybrid molecule of claim 1, wherein the hybrid molecule encodes an envelope protein comprising a membrane spanning domain (MSD), thereby preventing the subject from becoming infected with HIV-1.

22. A method for treating a subject infected with HIV-1, the method comprising administering to the subject in need thereof an effective amount of a composition comprising the hybrid molecule of claim 1, wherein the hybrid molecule encodes an envelope protein comprising a membrane spanning domain (MSD), thereby treating the subject infected with HIV-1.

23. A method for enhancing expression of an envelope protein in a cell, the method comprising expressing the hybrid molecule of claim 1 in a cell, thereby enhancing expression of the envelope protein in the cell.


SEQUENCE LISTING (7 sequences)

SEQ ID NO: 1 - protein, 5 aa, Artificial sequence
  Chemically synthesized
GYRPV

SEQ ID NO: 2 - protein, 5 aa, Artificial sequence
  Chemically synthesized
GYSPL

SEQ ID NO: 3 - protein, 5 aa, Artificial sequence
  Chemically synthesized
GIRPV

SEQ ID NO: 4 - protein, 3 aa, Artificial sequence
  Chemically synthesized
RPV

SEQ ID NO: 5 - protein, 5 aa, Artificial sequence
  Chemically synthesized
GYGPV

SEQ ID NO: 6 - protein, 13 aa, Artificial sequence
  Chemically synthesized
QGYRPVFSSPPSY

SEQ ID NO: 7 - protein, 13 aa, Artificial sequence
  Chemically synthesized
QGYRPVFSPPPSY